(19) United States
(12) Patent Application Publication
(10) Pub. No.: US 2004/0096881 A1
(43) Pub. Date: May 20, 2004

(54) eNOS mutants useful for gene therapy

(75) Inventors: Eric Blasko, Martinez, CA (US); Katalin Kauser, El Sobrante, CA (US); John F. Parkinson, Martinez, CA (US)

Correspondence Address: BERLEX BIOSCIENCES, PATENT DEPARTMENT, 2600 HILLTOP DRIVE, P.O. BOX 4099, RICHMOND, CA, 94804-0099, US

(73) Assignee: Schering Aktiengesellschaft, Berlin (DE)

(21) Appl. No.: 10/641,924

(22) Filed: Aug. 15, 2003

Related U.S. Application Data
(60) Provisional application No. 60/403,638, filed on Aug. 16, 2002.

(51) Int. Cl. C12Q 1/68; C07H 21/04; C12N 9/06

(52) U.S. Cl. 435/6; 435/69.1; 435/191; 435/320.1; 435/325; 536/23.2

(57) ABSTRACT

The present invention provides endothelial nitric oxide synthase (eNOS) polypeptide mutants, and polynucleotides encoding such polypeptide mutants, useful for gene therapy. In particular, the invention provides eNOS polypeptide mutants having one or more mutations in an amino acid sequence corresponding to a functional domain of a mammalian eNOS. More particularly, the invention provides eNOS polypeptide mutants having at least one mutation at a position corresponding to an amino acid residue in a calmodulin-binding site that is phosphorylated in mammalian cells, where the mutation is not an amino acid substitution to Ala or Asp in an eNOS polypeptide mutant having a single mutation that is at the phosphorylation site; and to polynucleotides encoding such polypeptide mutants. The present invention further provides prophylactic, diagnostic, and therapeutic methods of using such eNOS polypeptide mutants and polynucleotides.

This application claims the benefit of U.S. Provisional Application Serial No. 60/403,638, filed Aug. 16, 2002, which is incorporated herein by reference.

### FIELD OF THE INVENTION

The present invention relates to endothelial nitric oxide synthase (eNOS) polypeptide mutants, and polynucleotides encoding such polypeptide mutants, useful for gene therapy. In particular, the invention provides eNOS polypeptide mutants having one or more mutations in an amino acid sequence corresponding to a functional domain of a mammalian eNOS. More particularly, the invention relates to eNOS polypeptide mutants having at least one mutation at a position corresponding to an amino acid residue in a calmodulin-binding site that is phosphorylated in mammalian cells, where the mutation is not an amino acid substitution to Ala or Asp in an eNOS polypeptide mutant having a single mutation that is at the phosphorylation site. The present invention further relates to prophylactic, diagnostic, and therapeutic methods of using such eNOS polypeptide mutants and polynucleotides.

### BACKGROUND OF THE INVENTION

Endothelial nitric oxide synthase (eNOS, also called ecNOS or NOS3), and the nitric oxide (NO) generated by eNOS enzymatic activity, plays an important role in a variety of physiological processes including, e.g., angiogenesis, vasodilation, immune regulation, inhibition of platelet aggregation, and relaxation of smooth muscle. The regulation of eNOS activity involves the participation of a variety of secondary messenger molecules and their interaction with various functional areas on the polypeptide (see, e.g., Marletta, M. Trends in Biochem. Sciences (2001) 26:519-521).

The function and location of various functional domains of eNOS are well characterized and include, e.g., proceeding from the N-terminus to the C-terminus, a consensus site for myristoylation; sites for palmitoylation; an oxygenase domain; a calmodulin-binding site, and a reductase domain (see e.g., FIG. 1; also see e.g., Stuehr, D. J. Annu. Rev. Pharmacol. Toxicol. (1997) 37:339-359). A comparison of the eNOS sequence from a variety of species shows a high degree of sequence identity within each functional domain. In addition, consensus sequences for many of the functional domains are known. In response to various biological stimuli (e.g., cellular stimuli), wild-type eNOS is phosphorylated or dephosphorylated, in vitro or in vivo, by a number of specific kinases or phosphatases, at amino acid residues within the calmodulin-binding site and the reductase domain. Further, the phosphorylation levels of these sites contribute to the regulation of eNOS enzymatic activity (see, e.g., Fulton et al. Nature (1999) 399:597-601). In human wild-type eNOS (SEQ ID NO: 1), an amino acid substitution of the serine at amino acid residue Ser-1177 located in the reductase domain of a human wild-type eNOS (SEQ ID NO: 1) results in an eNOS polypeptide that is resistant to phosphorylation (and, consequently, to activation of eNOS activity) or that is a constitutively active (see e.g., WO 00/62605). Similar results are seen when an amino acid substitution of a corresponding amino acid residue is made in other species of eNOS s (see e.g., WO 00/62605).

A complex of calmodulin (CaM) and calcium (Ca⁺⁺) can bind efficiently to the eNOS calmodulin-binding site, and stimulate eNOS activity (e.g., NO production). Further, the binding of the CaM-Ca⁺⁺ complex to eNOS can be effected by the level of phosphorylation of a particular amino acid residue within the calmodulin-binding site. For example, when Thr-495 is phosphorylated, calmodulin-binding can be inhibited and/or the Ca⁺⁺ dependence of the calmodulin activation of eNOS can be inhibited. If phosphorylation at the Thr-495 is prevented, e.g. by specific kinase inhibitors or by changing Thr-495 to an Ala, eNOS activity can be stimulated (see e.g., Busse et al.)

Endothelial NO synthases are involved in a variety of activities as described herein, and aberrant expression and/or activity of these eNOS polypeptides, and/or aberrant amounts of NO produced by these enzymes, are associated with a variety of disease conditions. Thus, modulation of eNOS polypeptide levels and activity in cells clearly represents a useful therapeutic target.

### SUMMARY OF THE INVENTION

The present invention provides isolated endothelial nitric oxide synthase (eNOS) polypeptide mutants, polynucleotides encoding such polypeptides, and variants thereof, useful for gene therapy. In particular, the invention provides eNOS polypeptide mutants having one or more mutations in an amino acid sequence corresponding to a functional domain of a mammalian eNOS, where at least one mutation is at a position corresponding to an amino acid residue in a calmodulin-binding site that is phosphorylated in mammalian cells, and is not an amino acid substitution to Ala or Asp in an eNOS polypeptide mutant having a single mutation that is at the phosphorylation site.

In one aspect, the present invention provides an isolated human eNOS polypeptide mutant having a mutation at a position corresponding to position 495 of a human eNOS polypeptide, preferably the human eNOS encoded by SEQ ID NO: 1, where the mutation is not an amino acid substitution to Ala or Asp in an eNOS polypeptide mutant having a single mutation that is at a phosphorylation site of a calmodulin-binding site. In some aspects, the mutation corresponding to position 495 is an amino acid substitution to Gly, Val, Leu, Ile, Pro, Phe, Tyr, Trp, Met, Ser, Cys, Glu, Asn, Gin, Lys, Arg, or His and is preferably Val, Leu, or Ile, and most preferably Val. In some aspects, where the eNOS polypeptide mutant has more than one mutation, the mutation corresponding to position 495 is an amino acid substitution preferably to Ala or Val.

In another aspect, the present invention provides an isolated human eNOS polypeptide mutant having at least one mutation at a position corresponding to an amino acid residue, in a calmodulin-binding site of a mammalian eNOS, that is phosphorylated in mammalian cells; and further comprises at least one mutation at a position corresponding to an amino acid residue, in a reductase domain of a mammalian eNOS, that is phosphorylated in mammalian cells. In some aspects, the mutation in the calmodulin-binding domain is at a position corresponding to amino acid residue 495 of a human eNOS and is an amino acid substitution to Gly, Val, Leu, Ile, Pro, Phe, Tyr, Trp, Met, Ser, Cys, Glu, Asn, Gln, Lys, Arg, or His, and is preferably Val, Leu, or Ile, and most preferably Val; and the mutation in the reductase domain is at a position corresponding to amino acid residue 1177 of a human eNOS and is an amino acid substitution preferably to Asp. In some aspects, where the eNOS polypeptide mutant has more than one mutation, the mutation corresponding to position 495 is an amino acid substitution preferably to Ala or Val. Preferably, the human eNOS is the human eNOS encoded by SEQ ID NO: 1.

In another aspect, the present invention provides an isolated human eNOS polypeptide mutant having at least one mutation at a position corresponding to an amino acid residue, in a calmodulin-binding site of a mammalian eNOS, that is phosphorylated in mammalian cells, and further comprises at least one mutation at a position corresponding to an amino acid residue, at a myristoylation site of mammalian eNOS. In some aspects, the mutation in the calmodulin-binding domain is at a position corresponding to amino acid residue 495 of a human eNOS and is an amino acid substitution to Gly, Val, Leu, Ile, Pro, Phe, Tyr, Trp, Met, Ser, Cys, Glu, Asn, Gln, Lys, Arg, or His, and is preferably Val, Leu, or Ile, and most preferably Val; and the mutation at the myristoylation site is at a position corresponding to amino acid residue 2 of a human eNOS and is an amino acid substitution to Ala. In this aspect where the eNOS polypeptide mutant has more than one mutation, the mutation corresponding to position 495 is an amino acid substitution more preferably to Ala or Val. Preferably, the human eNOS is the human eNOS encoded by SEQ ID NO: 1.

In another aspect, the present invention provides an isolated human eNOS polypeptide mutant comprising: 1) at least one mutation at a position corresponding to an amino acid residue, in a calmodulin-binding site of a mammalian eNOS, that is phosphorylated in mammalian cells; 2) further comprises at least one mutation at a position corresponding to an amino acid residue, at a myristoylation site, of a mammalian eNOS; and 3) further comprises a mutation at a position corresponding to an amino acid residue, in a reductase domain of a mammalian eNOS, that is phosphorylated in mammalian cells. In some aspects, the mutation in the calmodulin-binding domain is at a position corresponding to amino acid residue 495 of a human eNOS and is an amino acid substitution to Gly, Val, Leu, Ile, Pro, Phe, Tyr, Trp, Met, Ser, Cys, Glu, Asn, Gln, Lys, Arg, or His and is preferably Val, Leu, or Ile, and most preferably Val; the mutation at the myristoylation site is at a position corresponding to amino acid residue 2 of a human eNOS and is an amino acid substitution to Ala; and the mutation in the reductase domain is at a position corresponding to amino acid 1177 of a human eNOS and is an amino acid substitution preferably to Asp. In this aspect where the eNOS polypeptide mutant has more than one mutation, the mutation corresponding to position 495 is an amino acid substitution more preferably to Ala or Val. Preferably, the human eNOS is the human eNOS encoded by SEQ ID NO: 1.

In another aspect, the phosphorylation of the eNOS polypeptide mutant of the present invention is increased or decreased, as compared to a reference eNOS polypeptide.

In another aspect, the Ca++ dependence of the eNOS polypeptide mutant of the present invention is decreased in Ca++-calmodulin mediated stimulation of the polypeptide, as compared to a reference eNOS polypeptide.

In another aspect, the eNOS polypeptide mutant of the present invention has an increased activity, as compared to a reference eNOS polypeptide.

In one aspect, the eNOS polypeptide mutant of the present invention has increased NO production, as compared to a reference eNOS polypeptide.

In one aspect, the eNOS polypeptide mutant of the present invention has increased reductase activity, as compared to a reference eNOS polypeptide.

In one aspect, the reference eNOS polypeptide is, or is derived from, the amino acid sequence of a human eNOS, preferably, the human eNOS encoded by SEQ ID NO: 1.

In another aspect, the invention provides an isolated eNOS polypeptide mutant that has an amino acid sequence that is substantially homologous to the amino acid sequence of a wild-type or mutant eNOS polypeptide. In one aspect, the invention provides an isolated eNOS polypeptide mutant having an amino acid sequence that has a 95-99% sequence identity to the amino acid sequence of a wild-type eNOS or mutant eNOS polypeptide of the present invention. Preferably, the starting eNOS polypeptide is a human eNOS polypeptide, and most preferably is, or is derived from, a human wild-type eNOS polypeptide, e.g., the human eNOS encoded by SEQ ID NO: 1.

In another aspect, the invention provides a polynucleotide encoding an eNOS polypeptide mutant of the present invention. In one aspect, the invention provides a recombinant vector having a polynucleotide encoding an eNOS polypeptide mutant of the present invention, where the polynucleotide is operably linked to at least one regulatory sequence such that the encoded polypeptide is expressed in cells.

In another aspect, the invention provides a pharmaceutical composition comprising an eNOS polypeptide mutant of the present invention. In another aspect, the invention provides a pharmaceutical composition comprising a polynucleotide encoding an eNOS polypeptide mutant of the present invention.

In another aspect, the invention provides a binding partner of an eNOS polypeptide mutant of the present invention. In one aspect, the binding partner is a polypeptide. In an additional aspect, the binding partner is an antibody or antigen-specific fragment.

In another aspect, the invention provides a method of modulating eNOS activity in a cell, comprising administering to a cell a polynucleotide encoding an eNOS polypeptide mutant of the present invention. In another aspect, the invention provides a method of modulating eNOS activity in a cell, comprising administering to a cell an eNOS polypeptide mutant of the present invention.

In another aspect, the invention provides a method of diagnosing a condition associated with aberrant eNOS activity, where the method comprises: 1) contacting a cell of a patient with a polynucleotide encoding an eNOS polypeptide mutant of the present invention; and 2) detecting a level of eNOS activity indicative of the condition.

In another aspect, the invention provides a prophylactic or therapeutic method of treating a condition associated with aberrant eNOS activity, where the method comprises administering to a patient in need of treatment an effective amount of an eNOS polypeptide mutant of the present invention.

In another aspect, the invention provides a prophylactic or therapeutic method of treating a condition associated with aberrant eNOS activity, where the method comprises administering to a patient in need of treatment an effective amount of a polynucleotide encoding an eNOS polypeptide mutant of the present invention, such that the polypeptide mutant is expressed in the patient.

In another aspect, the prophylactic and therapeutic methods of the invention further comprise, administering one or more angiogenic factors to a patient in need of treatment, before, during, or after the administering of an eNOS polypeptide mutant or polynucleotide encoding an eNOS polypeptide.

### BRIEF DESCRIPTION OF THE DRAWINGS

The foregoing and other objects of the present invention, the various features thereof, as well as the invention itself may be more fully understood from the following description, when read together with the accompanying drawings in which:

FIG. 1 is a diagram illustrating various functional domains of mammalian NOS. The functional domains include, but are not limited to, e.g., (proceeding from the N-terminus to the C-terminus) a consensus site for myristoylation; two sites for palmoylation; an oxidase domain; a calmodulin-binding site (e.g., amino acids 494-517 of human eNOS), which comprises a consensus sequence for phosphorylation (e.g., Thr-495 of human eNOS); and a reductase domain. Functional domains of NOS polypeptides also include, e.g., an autoinhibitory loop and a heme-binding site.

FIG. 2 is a histogram illustrating the stimulation of NO production in HEK 293 cells by eNOS polypeptide mutants having a single or double mutation, as compared to the wild-type human eNOS encoded by SEQ ID NO: 1 (WT). The eNOS polypeptide mutants having a single mutation, have an amino acid substitution to Asp (T495D), Ala (T495 A), or Val (T495V) at a position corresponding to Thr-495 of the human eNOS encoded by SEQ ID NO: 1. The eNOS polypeptide mutants having a double mutation, have a first amino acid substitution to Asp at a position corresponding to Ser-1177, and a second amino acid substitution to Asp (T495D+S1177D), Ala (T495AV+S1177D), or Val (T495V+S1177D) at a position corresponding to Thr-495 of the human eNOS encoded by SEQ ID NO: 1.

FIG. 3 is a histogram illustrating the stimulation of NO production in human aortic endothelium cells (HAEC) by eNOS polypeptide mutants having a single mutation, as compared the wild-type human eNOS encoded by SEQ ID NO: 1 (wild-type). The eNOS polypeptide mutants having a single mutation, have an amino acid substitution to Asp (T495D), Ala (T495A), or Val (T495V) at a position corresponding to Thr-495 of the human eNOS encoded by SEQ ID NO: 1.

### DETAILED DESCRIPTION OF THE INVENTION

The present invention provides endothelial nitric oxide synthase (eNOS) polypeptide mutants, polynucleotides encoding such polypeptides, and variants thereof, useful for gene therapy. In particular, the invention provides eNOS polypeptide mutants having one or more mutations in an amino acid sequence corresponding to a functional domain of a mammalian eNOS, where at least one mutation is at a position corresponding to an amino acid residue in a calmodulin-binding site that is phosphorylated in mammalian cells; and is not an amino acid substitution to Ala or Asp in an eNOS polypeptide mutant having a single mutation that is at a phosphorylation site of a calmodulin-binding site.

The present inventors have discovered that particular amino acid substitutions at the Thr-495 residue in the calmodulin-binding site of a human eNOS can, alone (where not Ala or Asp), or in combination with a second mutation at the Ser-1177, increase eNOS activity (e.g. NO production) in cells, as compared to the wild-type human eNOS (e.g., see Example 2); and that using such eNOS polypeptide mutants in gene therapy applications can ameliorate conditions associated with eNOS activity (as described in U.S. patent application serial No. 60/403,637, incorporated herein by reference).

Consequently, the eNOS polypeptides and methods of the present invention can be used to modulate the level of eNOS activity in cells, thereby providing a novel therapeutic approach for treating diseases and conditions associated with eNOS activity. For example, this novel approach targets the underlying pathophysiology of critical limb ischemia (CLI) through multiple mechanisms including, e.g.,: 1) the stimulation of angiogenesis; 2) the amelioration of microvascular dysfunction; 3) the restoration of vasomotor (vasodilator) activity of existing vessels; and 4) the remodeling/maturation of existing collaterals (arteriogenesis). The resulting improvement of blood flow and oxygen delivery to both skin and muscle is expected to relieve rest pain and heal ischemic ulcers.

Moreover, the eNOS polypeptide mutants of the present invention can be more efficacious than wild-type eNOS, due to the significantly higher specific activity of the mutant eNOS enzyme. In addition, the activity of eNOS polypeptides can be tightly regulated by calcium, and resistant to oxLDL and age. Consequently, in contrast to growth factors, toxicity due to “overdosing” could be negligible using the eNOS compositions of the present invention in gene therapy applications.

The references cited herein are incorporated by reference, in their entirety.

Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Standard reference works setting forth the general principles of recombinant DNA technology include Sambrook, J., et al. (1989) Molecular Cloning: A Laboratory Manual, 2d Ed., Cold Spring Harbor Laboratory Press, Planview, N.Y.; McPherson, M. J., Ed. (1991) Directed Mutagenesis: A Practical Approach, IRL Press, Oxford; Jones, J. (1992) Amino Acid and Peptide Synthesis, Oxford Science Publications, Oxford; Austen, B. M. and Westwood, O. M. R. (1991) Protein Targeting and Secretion, IRL Press, Oxford. Any suitable materials and/or methods known to those of ordinary skill in the art can be utilized in carrying out the present invention; however, preferred materials and/or methods are described. Materials, reagents and the like to which reference are made in the following description and examples are obtainable from commercial sources, unless otherwise noted.

As used herein, “polypeptide” refers to a full-length protein or fragment thereof, or peptide.

As used herein, “variant” with reference to a polypeptide or polynucleotide, refers to a polypeptide or polynucleotide that may vary in primary, secondary, or tertiary structure, as compared to a reference polypeptide or polynucleotide, respectively (e.g., as compared to a wild-type polypeptide or polynucleotide). For example, the amino acid or nucleic acid sequence may contain a mutation or modification that differs from a reference amino acid or nucleic acid sequence. In some embodiments, an eNOS variant may be a different isoform or polymorphism. Variants can be naturally-occurring, synthetic, recombinant, or chemically modified polypeptides or polynucleotides isolated or generated using methods well known in the art.

As used herein, “mutation” with reference to a polypeptide or polynucleotide, refers to a naturally-occurring, synthetic, recombinant, or chemical change or difference to the primary, secondary, or tertiary structure of a polypeptide or polynucleotide, as compared to a reference polypeptide or polynucleotide, respectively (e.g., as compared to a wild-type polypeptide or polynucleotide). Polypeptides and polynucleotides having such mutations can be isolated or generated using methods well known in the art.

As used herein, an “eNOS polypeptide mutant” or grammatical equivalents thereof (e.g., eNOS mutant, mutant eNOS, eNOS mutant polypeptide, mutant eNOS polypeptide) refers to an eNOS polypeptide, or variant thereof, having at least one variation or mutation in an amino acid residue corresponding to a position in a functional domain of a mammalian eNOS. In a preferred embodiment, the mutation is an amino acid substitution at a position corresponding to amino acid residue 495 of a human eNOS, where the amino acid substitution is not to an Ala or Asp in an eNOS polypeptide mutant having a single mutation that is at a phosphorylation site of a calmodulin-binding site. In other preferred embodiments, where the eNOS polypeptide mutant has more than one mutation, the mutation corresponding to position 495 is an amino acid substitution preferably to Ala or Val.

In another preferred embodiment, the activity of the eNOS polypeptide mutant is increased or decreased as compared to a reference eNOS polypeptide.

As used herein, a “functional domain” of an eNOS polypeptide is any amino acid residue, site, or region in the polypeptide associated with an eNOS activity, including but not limited to, e.g., a protein-binding domain (e.g., a calmodulin-binding domain, kinase-binding domain, or ligand-binding domain), phosphorylation site, myristolation site, reductase domain, or activation site.

As used herein, “eNOS activity” refers to any activity associated with the enzyme in cells including, but not limited to, e.g., NO production, calmodulin-binding, stimulating angiogenesis, ameliorating microvascular dysfunction, restoring vasomotor (vasodilator) activity of existing vessels, contributing to the remodeling/maturation of existing collaterals (arteriogenesis). An eNOS activity may also be any other biological or cellular activity associated with the polypeptide, and more particularly, any such activity associated with a functional domain of an eNOS. An eNOS activity may also be the modulation of an activity associated with the enzyme, including but not limited to, e.g., the modulation of any of the eNOS activities described herein or known in the art.

As used herein, “modulation”, with reference to an eNOS activity, refers to an increase, decrease, induction, or repression of such activity. In some embodiments, such increase, decrease, induction, or repression of eNOS activity is relative to a reference molecule, e.g., eNOS wild-type or mutant polypeptide.

As used herein, “disease”, “condition”, or “disorder” refers to an undesirable condition in a cell, tissue, or organ of a patient where eNOS activity can be modulated to ameliorate the condition. Endothelial NOS is involved in a variety of physiological processes including, but not limited to, e.g., angiogenesis, vasodilation, immune regulation, inhibition of platelet aggregation, and relaxation of smooth muscle. Thus, modulating eNOS activity in a cell, tissue or organ of a patient in need of treatment can ameliorate a disease, condition, or disorder as described herein.

As used herein, a “patient” is a mammal and is preferably a human.

eNOS Polypeptide Mutants

The present invention provides eNOS polypeptide mutants, polynucleotides encoding such polypeptides, and variants thereof, useful for gene therapy. In particular, the invention provides eNOS polypeptide mutants having one or more mutations in an amino acid sequence corresponding to a functional domain of a mammalian eNOS, where at least one mutation is at a position corresponding to an amino acid residue in a calmodulin-binding site that is phosphorylated in mammalian cells; and is not an amino acid substitution to Ala or Asp in an eNOS polypeptide mutant having a single mutation that is at the phosphorylation site. In some preferred embodiments, where the eNOS polypeptide mutant has more than one mutation, the mutation corresponding to position 495 is an amino acid substitution preferably to Ala or Val.

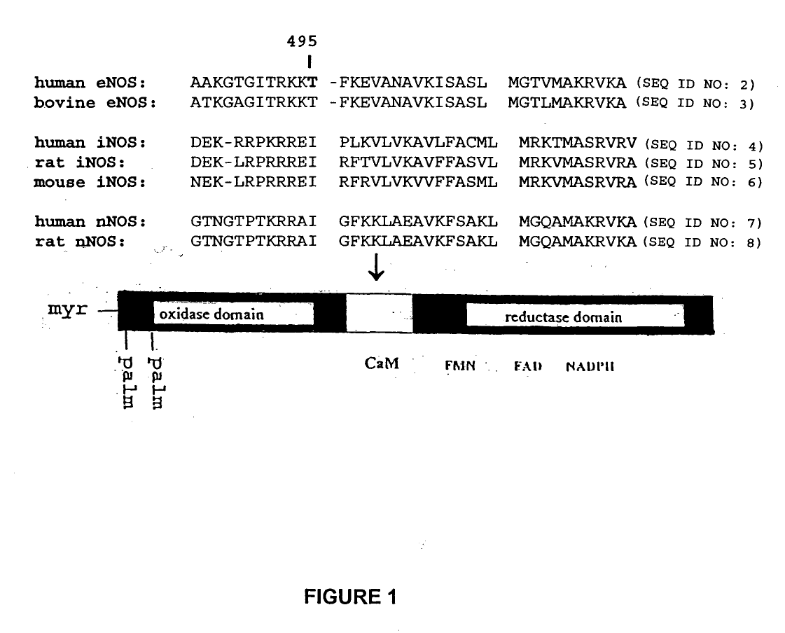

Functional domains of mammalian eNOS polypeptides are well characterized and include, e.g., proceeding from the N-terminus to the C-terminus, a consensus site for myristoylation; sites for palmitoylation; a calmodulin-binding site (e.g., amino acids 494-517 of a human eNOS), which comprises a consensus sequence for phosphorylation (e.g., Thr-495 of a human eNOS); a reductase domain and a consensus sequence for phosphorylation (e.g., Ser-1177 of a human eNOS). The location and characterization of these sites is well known (see e.g., Stuehr, D. J. Annu. Rev. Pharmacol. Toxicol. (1997) 37:339-359) (FIG. 1). In one embodiment, the eNOS polypeptide mutant of the present invention has one or more mutations within a calmodulin-binding domain, preferably at Thr-495, and a reductase domain, preferably at Ser-1177, where the mutation at Thr-495 is not an amino acid substitution to Ala or Asp in an eNOS polypeptide mutant having a single mutation that is at a phosphorylation site of a calmodulin-binding site.

In one embodiment, the eNOS polypeptide mutants of the present invention have a first mutation at a position corresponding to the Thr-495 residue of a human eNOS, where the mutation is not an amino acid substitution to Ala or Asp in an eNOS polypeptide mutant having a single mutation that is at a phosphorylation site of a calmodulin-binding site. In another embodiment, the eNOS polypeptide mutants of the present invention have a first mutation at a position corresponding to the Thr-495 residue of a human eNOS; and a second mutation at a position corresponding to the Ser-1177 residue of a human eNOS. In another embodiment, the eNOS polypeptide mutants of the present invention have a first mutation at a position corresponding to the Thr-495 residue of human eNOS; a second mutation at a position corresponding to the Ser-1177 residue of a human eNOS; and a third mutation at a position corresponding to the Gly-2 of a human eNOS.

Mutations of the invention can be any of a variety of types, including, e.g., one or more amino acid additions, substitutions, deletions, insertions, modifications, inversions, fusions, or truncations, or a combination of any of these, and can be generated synthetically, chemically, recombinantly, or by known methods. In preferred embodiments of the eNOS polypeptide mutants of the present invention, a mutation at a position corresponding to the Thr-495 of a human eNOS is an amino acid substitution to Gly, Val, Leu, lie, Pro, Phe, Tyr, Trp, Met, Ser, Cys, Glu, Asn, Gln, Lys, Arg, or His, and is preferably to Val, Leu, or Ile, and is most preferably to Val; a mutation at a position corresponding to Ser-1177 of a human eNOS is an amino acid substitution preferably to Asp, and is preferably to Ala; and a mutation at a position corresponding to Gly-2 of human eNOS is an amino acid substitution to Ala. In some preferred embodiments, where the eNOS polypeptide mutant has more than one mutation, the mutation corresponding to position 495 is an amino acid substitution preferably to Ala or Val

In one embodiment, the eNOS polypeptide mutant of the present invention comprises, in addition to a mutation at the Thr-495 site (indicated in bold type below), one or more additional mutated amino acid residues in the calmodulin-binding site, DPWKGSAAKGTGITRKKTFKEVANAVKISASLMGTVMAKRVKATI (SEQ ID NO:1, amino acids 478522). The comparable sequence in other species can be slightly different, particularly in residues that are N-terminal to the phosphorylation site. Each amino acid in this motif can be changed individually to any of the other 19 natural amino acids, or to a non-natural amino acid. In some embodiments, the mutation is not a conservative one, e.g., Gly/Ala, Val/Ile/Leu, Asp/Glu, Lys/Arg, Asp/Gln, Thr/Ser or Phe/Trp/Tyr. In one embodiment, starting with an eNOS polypeptide (starting polypeptide) a first mutation is introduced at a position corresponding to a phosphorylation site of a calmodulin-binding domain and the polypeptide mutant assayed for eNOS activity, as compared to a reference eNOS polypeptide (e.g., the starting polypeptide or other eNOS polypeptide, including a wild-type eNOS polypeptide). For example, a single mutant can be selected which exhibits a higher amount of eNOS activity (e.g., NO production), as compared to the starting eNOS polypeptide. After this first mutation has been made and characterized, the process can be repeated to generate an eNOS polypeptide having a double mutation, and can be further repeated to generate eNOS polypeptide mutants having additional mutations as described herein. Any number of mutations can be made using known methods.

Methods of generating polypeptide mutants (e.g., mutating the nucleic acid which encodes them) are standard and well known in the art. These methods include, e.g., homologous recombination, site-directed mutagenesis, cassette mutagenesis, and PCR-based mutagenesis (see, e.g., Sambrook et al., Molecular Cloning, CSH Press (1989); Kunkel et al. (1985) PNAS 82, 488-492; and Lee et al. (2001) J. Biol. Chem. Dec 21; 276(51):47930-6). The starting material for such mutations can be, e.g., an eNOS cDNA from any, e.g., human, mouse, guinea pig, dog, bovine, porcine, rabbit, rat, ovine, equine, non-human primate, or other animal.

In preferred embodiments, the eNOS polypeptide mutants of the present invention exhibit an increase or decrease in an eNOS activity, as compared to a reference eNOS polypeptide (e.g., NO production). In another embodiment, the eNOS polypeptide mutants of the present invention can exhibit an increase or decrease in one or more eNOS activities, and the level of increase or decrease in activity is relative to a reference eNOS polypeptide. Mutated polypeptides of the invention can be characterized by assaying for any of the eNOS activities described herein, using standard assays.

For example, in response to various stimuli (e.g., cellular stimuli), in vitro or in vivo, eNOS is phosphorylated or dephosphorylated, by specific kinases or phosphatases, at, e.g., Thr-495 and/or Ser-1177 of a human eNOS (or at comparable residues for other species). In preferred embodiments, an eNOS polypeptide mutant of the present invention exhibits increased NO production, binding or affinity of CaM for the CaM binding site of eNOS, and/or exhibits a reduced dependence on Ca⁺⁺ CaM-mediated activation an eNOS. Any of these and other eNOS activities described herein and known in the art, including activities that are indirectly mediated by NO produced by the enzyme, are activities that may be modulated by the eNOS compositions and methods of the present invention.

eNOS is found in, e.g., vascular endothelium, cardiac myocytes, blood platelets, and various types of cells of the immune system (e.g., T-cells, neutrophils, and monocytes), and converts L-Arg to NO, a gaseous messenger molecule that is involved in, and/or serves a regulatory function in many physiological responses. Further, eNOS binds to calmodulin, which in conjuction with Ca⁺⁺, activates eNOS enzymatic activity. Various eNOS-associated activities, include those activities which are directly and/or indirectly mediated by NO produced by the enzyme. Such eNOS activities include, but are not limited to, e.g., stimulation of angiogenesis (normal or impaired, e.g., as a result of ischemia); stimulation of vasodilation; stimulation of collateral vessel development; enhancement of peripheral limb blood flow; inhibition of limb necrosis (e.g., in critical limb ischemia, or CLI); enhanced wound healing; inhibition of smooth muscle contraction; inhibition or prevention of platelet adhesion and aggregation (which can lead to, e.g., inhibition of thrombus formation); mediation of the protective effects of elevated HDL on the cardiovascular system; stimulation of endothelial cell proliferation and migration; inhibition of leukocyte activation and adhesion, chemokine expression, or smooth muscle proliferation; suppression of myocardial contraction; regulation of an immune response; and scavenging of superoxide anion. Methods of assaying for these and other eNOS activities are well known to those of skill in the art.

For example, standard methods to assay the phosphorylation and/or the degree of phosphorylation of an eNOS polypeptide, include an in vitro method in which the protein (e.g., produced recombinantly in E. coli or partially or completely purified from a natural source) is incubated with a kinase, such as an AMP-activated kinase (AMPK) or protein kinase C (PKC), or with a phosphatase.

The eNOS polypeptides, or tryptic digests thereof, can then be analyzed using standard methods, such as gel electrophoresis or column chromatography coupled with autoradiography or immunoblotting with an antibody specific for a given phosphopeptide. For these and other assays, see e.g. WO00/28076; WO00/62605; WO00/62605, Michell et al. (2001) The Journal of Biological Chemistry 276, 17,625-628; and Fleming et al. (2001) Circulation Research 88, 68e-75e.

Other methods to measure various activities which are dependent, directly or indirectly, on the expression of eNOS, either in vitro and in vivo, include the following: (1) measuring L-[³H]-citrulline production, either in the presence or absence of calmodulin (CaM) (see, e.g., Balligand et al. (1995) J. Biol. Chem. 270, 14,582-586; Bredt et al. (1990) Proc. Natl. Acad. Sci. USA 87, 682-685; and Fleming et al. (2001) Circulation Research 88, 68e-75e). For example, recombinant eNOS can be co-expressed with CaM (see, e.g., Rodriguez-Crespo et al. (1996) Arch. Biochem. Biophys. 336, 151-156), and assayed in the presence of variable amounts of added EGTA (as described, e.g., in WO00/28076); (2) measuring the ability to bind to calmodulin (as described, e.g., in Fleming et al. (2001) Circulation Research 88, 68e-75e); (3) measuring, in intact cells, the time dependent and N′-nitro-L-arginine-sensitive accumulation of cGMP (see, e.g., Fleming et al. (1998) Circ. Res. 82, 686-695); (4) measuring reductase activity, e.g., NADPH-dependent reductase, cytochrome c reductase activity, or 2,6-dichlorophenolindophenol (DCIP) reduction (see, e.g., WO00/62605; WO00/62605; Martasek et al. (1999) Methods Enzymol. 301, 70-78; Masters et al. (1967) Methods Enzymol. 10, 565-573); (5) measuring the effect on smooth muscle contraction (see, e.g., Furchgott and Zawadzki (1980) Nature 288: 373-376); (6) measuring the effect on platelet function; (7) measuring the release of NO (assayed as nitrite, NO₂—) from cells, determined by chemiluminescence or by hemoglobin capture (see, e.g., WO00/62605; WO00/62605; Sessa (1995) J. Biol. Chem. 270, 17,641-644; Kelm et al. (1988) Biochem. Biophys. Res. Commun. 154, 236-244); (8) measuring the inhibition of limb necrosis, as evidenced by increased capillary density or vasomotor reactivity in the collateral vessel-dependent ischemic limb (see, e.g., Murohara et al. (1998) J. Clin. Invest., 101, 2567-2568); (9) measuring enhancement of wound healing, endothelial cell migration, proliferation or differentiation (see, e.g., Lee et al., (1999) Am J. Physiol. 277, H 1600-1608); or (10) measurement of erectile dysfunction. See also the Examples herein, which illustrate assays for eNOS activity (e.g., NO production).

Animal models for testing eNOS activity are standard and well-known in the art. See, e.g., to test angiogenesis and revascularization, Murohara et al. (1998 ibid); Couffinhal et al. (1998); Am J. Pathol 152, 1667-1669; Couffinhal et al., (1999) Circulation 99, 3188-3198; and Examples herein. Such assays include, e.g., mouse and rabbit models of surgical hindlimb ischemia, e.g., in which the surgery is performed in a eNOS knockout mouse. Methods to measure hindlimb blood flow and capillary density are also well-known (see, e.g., Murohara et al. (ibid); Couffinhal et al. (1998); Am J. Pathol 152, 1667-1669; Couffinhal et al., (1999) Circulation 99, 3188-3198; and Examples herein).

An eNOS polypeptide of the present invention may be a recombinant polypeptide, a natural polypeptide or a synthetic or semi-synthetic polypeptide, or combinations thereof, preferably a recombinant polypeptide.

The polypeptides of the present invention are preferably provided in an isolated form, and may be purified, e.g. to homogeneity. The term “isolated,” when referring, e.g., to a polypeptide or polynucleotide, means that the material is removed from its original environment (e.g., the natural environment if it is naturally-occurring), and isolated or separated from at least one other component with which it is naturally associated. For example, a naturally-occurring polypeptide present in its natural living host is not isolated, but the same polypeptide, separated from some or all of the coexisting materials in the natural system, is isolated. Such polypeptides could be part of a composition, and still be isolated in that such a composition is not part of its natural environment.

A fragment or variant of an eNOS polypeptide of the present invention preferably retains substantially at least one eNOS activity. Such eNOS activity can be, e.g., any of those described herein, and includes having the ability to react with an antibody, i.e., having an epitope-bearing peptide, particularly one which comprises one or more mutations of the invention.

Polypeptide fragments of the invention may be of any size that is compatible with the invention, e.g., useful for gene therapy. The fragments may range in size from the smallest specific epitope (e.g., about 6 amino acids) to a nearly full-length gene product (e.g., a single amino acid shorter than the full-length polypeptide). For example, a polypeptide of the invention may comprise at least about 10, 25, 50, 100, 200, 300, 400, 500, 600, 800, 1000, or 1200 amino acids.

Fragments of the polypeptides of the present invention may be employed, e.g., for producing the corresponding full-length polypeptide by peptide synthesis, e.g., as intermediates for producing the full-length polypeptides; for inducing the production of antibodies or antigen-binding fragments; as “query sequences” for the probing of public databases, or the like.

Furthermore, a polypeptide fragment which encompasses a mutation of the invention may be used as an eNOS antagonist. Without wishing to be bound to any particular mechanism, it is proposed that such a mutant-containing peptide fragment, particularly one which exhibits an increased affinity or binding to calmodulin compared to that of wild-type eNOS, can act to “sop up” calmodulin in a cell, and thus to inhibit calmodulin-induced activation of eNOS in the cell. The degree of inhibition can range from partial to complete inhibition.

A variant of a polypeptide of the invention (e.g., a variant of human eNOS polypeptide which is already altered in the Thr-495 site and/or other sites in the calmodulin-binding domain) may be, e.g., (i) one in which one or more of the amino acid residues are substituted with a conserved or non-conserved amino acid residue (preferably a conserved amino acid residue) and such a substituted amino acid residue may or may not be one encoded by the genetic code, or (ii) one in which one or more of the amino acid residues includes a substituent group, or (iii) one in which the polypeptide is fused with another compound, such as a compound to increase the half-life of the polypeptide (for example, polyethylene glycol), or (iv) one in which additional amino acids are fused to the polypeptide, such as a leader or secretory sequence or a sequence which is employed for purification of the polypeptide, commonly for the purpose of creating a genetically engineered form of the protein that is susceptible to secretion from a cell, such as a transformed cell. The additional amino acids may be from a heterologous source, or may be endogenous to the natural gene.

Variant polypeptides belonging to type (i) above include, e.g., muteins, polypeptide mutants and derivatives. A variant polypeptide can differ in amino acid sequence by, e.g., one or more additions, substitutions, deletions, insertions, inversions, fusions, and truncations or a combination of any of these. For example, conservative amino acid substitutions, which are well-known to those of skill in the art, generally do not lead to a change in protein function.

Variant polypeptides belonging to type (ii) above include, e.g., modified polypeptides. Known polypeptide modifications include, but are not limited to, glycosylation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formatin, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in many basic texts, such as Proteins—Structure and Molecular Properties, 2nd ed., T. E. Creighton, W. H. Freeman and Company, New York (1993). Many detailed reviews are available on this subject, such as by Wold, F., Posttranslationail Covalent Modification of Proteins, B. C. Johnson, Ed., Academic Press, New York 1-12 (1983); Seifter et al. (1990) Meth. Enzymol. 182:626-646 and Rattan et al. (1992) Ann. N.Y. Acad. Sci. 663:48-62.

Variant polypeptides belonging to type (iii) are well-known in the art and include, e.g., PEGylation or other chemical modifications.

Variants polypeptides belonging to type (iv) above include, e.g., preproteins or proproteins which can be activated by cleavage of the proprotein portion to produce an active mature polypeptide. Variants include a variety of hybrid, chimeric or fusion polypeptides. Typical examples of such variants are discussed elsewhere herein.

Many other types of variants are known to those of skill in the art. For example, as is well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications or variations can occur anywhere in a polypeptide, including the peptide backbone, the amino acid side-chains and the amino or carboxyl termini. The same type of modification may be present in the same or varying degree at several sites in a given polypeptide. Also, a given polypeptide may contain more than one type of modification. Blockage of the amino or carboxyl group in a polypeptide, or both, by a covalent modification, is common in naturally-occurring and synthetic polypeptides. For instance, the aminoterminal residue of polypeptides made in E. coli, prior to proteolytic processing, is often N-formylmethionine. The modifications can be a function of how the protein is made. For recombinant polypeptides, for example, the modifications are determined by the host cell posttranslational modification capacity and the modification signals in the polypeptide amino acid sequence. Accordingly, when glycosylation is desired, a polypeptide can be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

Variant polypeptides can exhibit an increase or decrease in one or more eNOS activities, where the increase or decrease of an eNOS activity is relative to the level of activity of a reference eNOS polypeptide.

As mentioned, the eNOS polypeptide mutants of the present invention include mutants in which one or more amino acids are modified in addition to the Thr-495 residue and/or other sites in the calmodulin-binding domain. That is, the additional mutation(s) lie in other functional domains of the eNOS polypeptide. For example, one or more mutations can be introduced into one or more of the catalytic domains (e.g., the oxidase or reductase domain), or the regulatory regions (e.g., the myristoylation site, the autoinhibitory loop, or the Ser phosporylation site which lies near the C-terminal region of the molecule, or any of the functional domains described elsewhere herein). The additional mutations can be any of the types of mutations described herein.

Exemplary additional mutations include, e.g., a substitution of the Ser phosphorylation site at residue 1177 of a human eNOS with another amino acid, such as Asp (see, e.g., WO00/62605), or a mutation at the myristoylation site of a human eNOS, such as a substitution of the Ala at residue 2 with another amino acid, e.g., a Gly (see, e.g., Sessa et al, (1993). Circulation Research 72, 921-924).

eNOS polypeptides mutated in the myristoylation site can be localized in the cytoplasm of a cell rather than at the membrane. Such mutants can be resistant (as compared to a wild-type eNOS) to pathological stimuli (e.g.,, oxLDL) which downregulate eNOS NO production. This property can be advantageous for use of the eNOS polypeptide mutant (or polynucleotide encoding it) in the treatment of conditions such as atherosclerosis, peripheral limb ischemia, or CLI, in which such external pathological stimuli exists.

In a preferred embodiment, a human eNOS polypeptide of the present invention comprises an amino acid substitution corresponding to a position at Thr-495 (e.g., to Ala, Val, Leu or Ile, preferably Ala or Val); and/or an amino acid substitution corresponding to a position at Ser-1177 (e.g, preferably to Asp); and/or an amino acid substitution corresponding to position Gly-2 (e.g., to Ala), where the double or triple mutant exhibits greater eNOS activity, as compared to a reference eNOS polypeptide (e.g., a wild-type eNOS, or other eNOS polypeptide mutant).

The eNOS polypeptide mutants of the invention also include polypeptides that have varying degrees of sequence homology (identity) to a wild-type eNOS or mutant eNOS polypeptide of the present invention. In one embodiment, the polypeptides are substantially homologous to an eNOS polypeptide of the present invention, or show substantial sequence homology (sequence identity) thereto. Thus, polypeptides, and fragments thereof, within the present invention may contain amino acid sequences which show at least about 65-70% sequence homology (identity) to a wild-type eNOS or mutant eNOS polypeptide of the invention, preferably about 70-75%, 75-80%, or 80-85%, 85-90% sequence homology (identity) thereto, and most preferably about 90-95% or 95-99% sequence homology (identity) thereto. The invention also encompasses polypeptides having a lower degree of sequence identity, but having sufficient similarity so as to exhibit one or more eNOS activities.

In accordance with the present invention, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity =100 [1−(C/R)]

wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., when aligning a second sequence to the amino acid sequences herein having 91 amino acid residues, at least 30, preferably at least 35, more preferably at least 45, even more preferably at least 55, and even more preferably at least 65, 70, 80 and 90 amino acid residues are aligned).

The description herein for percent identity or percent homology is intended to apply equally to nucleotide or amino acid sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

A preferred, non-limiting example of such a mathematical algorithm is described in Karlin et al. (1993) Proc. Natl. Acad. Sci. USA 90:5873-5877. Such an algorithm is incorporated into the NBLAST and XBLAST programs (version 2.0) as described in Altschul et al. (1997) Nucleic Acids Res. 25:3389-3402. When utilizing BLAST and Gapped BLAST programs, the default parameters of the respective programs (e.g., NBLASST) can be used. In one embodiment, parameters for sequence comparison can be set at score=100, wordlength-12, or can be varied (e.g., W=5 or W=20).

In a preferred embodiment, the percent identity between two amino acid sequences is determined using the Needleman et al. (1970) (J. Mol. Biol. 48:444-453) algorithm which has been incorporated into the GAP program in the GCG software package using either a BLOSUM 62 matrix or a PAM250 matrix, and a gap weight of 16, 14, 12, 10, 8, 6, or 4 and a length weight of 1, 2, 3, 4, 5 or 6. In yet another preferred embodiment, the percent identity between two nucleotide sequences is determined using the GAP program I the GCG software package (Devereux et al. (1984) Nucleic Acids Res. 12 (1):387) using a NWSgapdna. CMP matrix and a gap weight of 40, 50, 60, 70, or 80 and a length weight of 1, 2, 3, 4, 5 or 6.

Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis et al. (1994) Comput. Appl. Biosci. 10:3-5; and FASTA described in Pearson et al. (1988) PNAS85:2444-8.

In accordance with the present invention, the term “substantially homologous,” when referring to a protein sequence, means that the amino acid sequences are at least about 90-95% or 97-99% or more identical. A substantially homologous amino acid sequence can be encoded by a nucleic acid sequence hybridizing to the nucleic acid sequence, or portion thereof, of a sequence encoding a mutant polypeptide of the invention, under conditions of high stringency.

Conditions of “high stringency,” as used herein, means, for example, incubating a blot overnight (e.g., at least 12 hours) with a long polynucleotide probe in a hybridization solution containing, e.g., about 5×SSC, 0.5% SDS, 100 μg/ml denatured salmon sperm DNA and 50% formamide, at 42° C. Blots can be washed at high stringency conditions that allow, e.g., for less than 5% bp mismatch (e.g., wash twice in 0.1×SSC and 0.1% SDS for 30 min at 65° C.), thereby selecting sequences having, e.g., 95% or greater sequence identity.

Other non-limiting examples of high stringency conditions include a final wash at 65° C. in aqueous buffer containing 30 mM NaCl and 0.5% SDS. Another example of high stringent conditions is hybridization in 7% SDS, 0.5 M NaPO₄, pH 7, 1 mM EDTA at 50° C., e.g., overnight, followed by one or more washes with a 1% SDS solution at 42° C. Whereas high stringency washes can allow for less than 5% mismatch, reduced or low stringency conditions can permit up to 20% nucleotide mismatch. Hybridization at low stringency can be accomplished as above, but using lower formamide conditions, lower temperatures and/or lower salt concentrations, as well as longer periods of incubation time.

As used with respect to the polypeptides (and polynucleotides) of the present invention, the term fragment refers to a sequence that is a subset of a larger sequence (i.e., a continuous or unbroken sequence of residues within a larger sequence).

The polypeptides of the present invention may originate from cells and tissues of any species of mammal, e.g., mouse, rat, guinea pig, rabbit, farm animals, such as bovine, ovine or porcine, pets, such as dogs, equine, non-human primate, or other animal, or humans, but are preferably originate from human cells. The sequence of eNOS is known for many species, e.g., human (Janssens et al. (1992) J. Biol. Chem. 267, 14,519-522), bovine (SEQ ID NO: 2 of U.S. Pat. No. 5,498,539). See e.g., Dog (genbank ACCESSION AF143503) and guinea pig (genbank ACCESSION AF146041).

In any given mammal, eNOS polypeptides may be found in a variety of tissues. Methods of determining the tissue or cellular location of such polypeptides are standard and include, e.g., standard methods of immunohistochemistry. eNOS polypeptides are found in, e.g., vascular endothelium, cardiac myocytes, blood platelets, and various cells of the immune system, such as e.g., T-cells, neutrophils, and monocytes.

Polynucleotides Encoding eNOS Polypeptide Mutants

The invention also includes polynucleotides, and fragments thereof, encoding the eNOS polypeptide mutants of the present invention. The invention also includes polynucleotides that code without interruption for an eNOS polypeptide mutant of the present invention. A polynucleotide that “codes without interruption” refers to a polynucleotide having a continuous open reading frame (“ORF”) as compared to an ORF which is interrupted by introns or other noncoding sequences.

A polynucleotide of the present invention may be a recombinant polynucleotide, a natural polynucleotide, or a synthetic or semi-synthetic polynucleotide, or combinations thereof. As used herein, the terms polynucleotide, oligonucleotide, oligomer and nucleic acid are interchangeable. Thus, reference to a “polynucleotide” can encompass fragments, such as oligonucleotides, of a full-length polynucleotide.

As used herein, the term “gene” means a segment of DNA involved in producing a polypeptide chain; it may include regions preceding and following the coding region (leader and trailer) as well as intervening sequences (introns) between individual coding segments (exons). Of course, cDNAs lack the corresponding introns. The invention includes isolated genes (e.g., genomic clones) which encode polypeptides of the invention.

Polynucleotides of the invention may be RNA, PNA, or DNA, e.g., cDNA, genomic DNA, and synthetic or semi-synthetic DNA, or combinations thereof. The DNA may be triplex, double-stranded or single-stranded, and if single stranded, may be the coding strand or non-coding (anti-sense) strand. It can comprise hairpins or other secondary structures. The RNA includes oligomers (including those having sense or antisense strands), mRNAs, polyadenylated RNA, total RNA, single strand or double strand RNA, or the like. DNA/RNA duplexes are also encompassed by the invention.

The polynucleotides, and fragments thereof, of the present invention may be of any size that is compatible with the invention, e.g., of any desired size that is effective to achieve a desired specificity when used as a probe. Polynucleotides may range in size, e.g., from the smallest specific probe (e.g., about 10-12 nucleotides) to greater than a full-length cDNA, e.g., in the case of a fusion polynucleotide or a polynucleotide that is part of a genomic sequence; fragments may be as large as, e.g., one nucleotide shorter than a full-length cDNA. For example, a polynucleotide of the invention many comprise at least about 8, 10, 12, 14 or 15 contiguous nucleotides, e.g., about 15 continuous nucleotides.

A fragment of a polynucleotide according to the invention may be used, e.g., as a hybridization probe, as discussed elsewhere herein. A fragment of a polynucleotide may also be useful as a starting point for cassette mutagenesis. Cassette mutagenesis (see e.g., Lee et al. (2001) supra) allows for the introduction of many mutations into a sequence at the same time. Individual clones can then be expressed and the desired phenotype selected by a screening method and the product sequenced. For example, full-length eNOS mutants can be expressed in E. coli and mutants selected by adsorption onto a calmodulin affinity column, eluted and then Western blotted. The sequence of the mutants that bind can then be determined using standard sequencing protocols. Similarly, the mutated sequences can be introduced into an expression system that expresses the motif as an epitope on e.g., phage display. Phage can be bound to a calmodulin affinity column, selected, and sequenced. A peptide library approach can also be used as the sequence of the eNOS calmodulin-binding domain is amenable to total synthesis.

Many types of variants of polynucleotides are encompassed by the invention including, e.g., (i) one in which one or more of the nucleotides is substituted with another nucleotide, or which is otherwise mutated; or (ii) one in which one or more of the nucleotides is modified, e.g., includes a subtituent group; or (iii) one in which the polynucleotide is fused with another compound, such as a compound to increase the half-life of the polynucleotide; or (iv) one in which additional nucleotides are covalently bound to the polynucleotide, such a sequences encoding a leader or secretory sequence or a sequence which is employed for purification of the polypeptide. The additional nucleotides may be from a heterologous source, or may be endogenous to the natural gene.

A polynucleotide of the invention may have a coding sequence which is a naturally or non-naturally-occurring allelic variant of a coding sequence encompassed by the sequence of wild-type eNOS. As is known in the art, an allelic variant is an alternate form of a polynucleotide sequence which may have a substitution, deletion or addition of one or more nucleotides, which in general does not substantially alter the function of the encoded polypeptide.

Other variant sequences, located in a coding sequence or in a regulatory sequence, may affect (e.g., enhance or decrease) the production of, or the function or activity of, an eNOS polypeptide mutant of the invention.

Polynucleotide variants belonging to type (ii) above include, e.g., modifications such as the attachment of detectable markers (avidin, biotin, radioactive elements, fluorescent tags and dyes, energy transfer labels, energy-emitting labels, binding partners, etc.) or moieties which improve expression, uptake, cataloging, tagging, hybridization, detection, and/or stability. The polynucleotides can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; 5,478,893.

Polynucleotide variants belonging to type (iii) above are well known in the art and include, e.g., various lengths of polyA⁺ tail, 5′cap structures, and nucleotide polypeptide mutants, e.g., inosine, thionucleotides, or the like.

Polynucleotide variants belonging to type (iv) above include, e.g., a variety of chimeric, hybrid or fusion polynucleotides. For example, a polynucleotide of the invention can comprise a coding sequence and additional non-naturally-occurring or heterologous coding sequence (e.g., sequences coding for leader, signal, secretory, targeting, enzymatic, fluorescent, antibiotic resistance, and other functional or diagnostic peptides); or a coding sequence and non-coding sequences, e.g., untranslated sequences at either a 5′ or 3′ end, or dispersed in the coding sequence, e.g., introns.

More specifically, the present invention includes polynucleotides where the coding sequence for an eNOS polypeptide mutant is fused in the same reading frame to another polypeptide sequence encoded by the polynucleotide (e.g., a heterologous polypeptide sequence) to produce a fusion eNOS polypeptide mutant. Polypeptide sequences which can be fused in this manner are, e.g., sequences that aid in expression and secretion of a polypeptide from a host cell is a leader sequence which functions as a secretory sequence for controlling transport of a polypeptide from the cell and/or a transmembrane anchor sequence which facilitates attachment of the polypeptide to a cellular membrane. A polypeptide having a leader sequence is a preprotein and may have the leader sequence cleaved by the host cell to form a mature form of the polypeptide. The polynucleotides may also encode for a proprotein which is the mature protein plus additional N-terminal amino acid residues. A mature protein having a prosequence is a proprotein and is generally an inactive form of the protein, and once the prosequence is cleaved an active protein remains. Polynucleotides of the present invention may also have a coding sequence fused in-frame to a marker sequence that allows for identification and/or purification of the polypeptide of the present invention. The marker sequence may be, e.g., a hexa-histidine tag (e.g., as supplied by a pQE-9 vector) to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (see, e.g., Wilson, I., et al., Cell, 37:767 (1984)).

Other types of polynucleotide variants will be evident to one of skill in the art. For example, the nucleotides of a polynucleotide can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAse H, improved in vivo stability (see, e.g., U.S. Pat. No. 5,378,825). Any desired nucleotide or nucleotide polypeptide mutant can be incorporated, e.g., 6-mercaptoguanine, 8-oxo-guanine.

Also, polynucleotides of the invention may have a coding sequence derived from another genetic locus of an organism, providing it has a substantial homology to a mammalian wild-type eNOS polypeptide or to one from another organism (e.g., an ortholog).

Polynucleotides of the present invention can be labeled according to any desired method. For example, a polynucleotide of the present invention can be labeled using radioactive tracers such as, e.g., ³²P, ³⁵S, ³H, or ¹⁴C. The radioactive labeling can be carried out according to any method, such as, for example, terminal labeling at the 3′ or 5′ end using a radiolabeled nucleotide, polynucleotide kinase (with or without dephosphorylation with a phosphatase) or a ligase (depending on the end of the polynucleotide to be labeled). A non-radioactive labeling can also be used, combining a polynucleotide of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

A polynucleotide of the invention may comprise a sequence which has a sequence identity of at least about 65-100% (e.g., at least about 70-75%, 80-85%, 90-95% or 97-99%) to, or which is substantially homologous to, or which hybridizes under conditions of high stringency to, a nucleotide sequence encoding a wild-type eNOS or mutant eNOS polypeptide of the present invention.

The term “substantially homologous,” when referring to polynucleotide sequences, means that the nucleotide sequences are at least about 90-95% or 97-99% or more identical to a polynucleotide encoding a wild-type or mutant eNOS polypeptide of the present invention.

Expression of eNOS Polypeptides and Assays for eNOS Activity

The present invention also relates to recombinant constructs that contain vectors plus polynucleotides of the present invention. Such constructs comprise a vector, such as a plasmid or viral vector, into which a polynucleotide sequence of the invention has been inserted, in a forward or reverse orientation.

Large numbers of suitable vectors are known to those of skill in the art, and many are commercially available. The following vectors are provided by way of example; Bacterial: pQE70, pQE60, pQE-9 (Qiagen), pBS, pD10, phagescript, psiX174, pBluescript SK, pBSKS, pNH8A, pNH16a, pNH18A, pNH46A (Stratagene); pTRC99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as it is replicable and viable in the host.

In a preferred embodiment, the vector is an expression vector, into which a polynucleotide sequence of the invention is inserted so as to be operatively linked to an appropriate expression control (regulatory) sequence(s) (e.g., promoters and/or enhancers) which directs mRNA synthesis. Appropriate expression control sequences, e.g., regulatable promoter or regulatory sequences known to control expression of genes in prokaryotic or eukaryotic cells or their viruses, can be selected for expression in prokaryotes (e.g., bacteria), yeast, plants, mammalian cells or other cells. Preferred expression control sequences are derived from highly-expressed genes, e.g., from operons encoding glycolytic enzymes such as 3-phosphoglycerate kinase (PGK), α-factor, acid phosphatase, or heat shock proteins, among others. Such expression control sequences can be selected from any desired gene, e.g using CAT (chloramphenicol transferase) vectors or other vectors with selectable markers. Two appropriate vectors for such selection are pKK232-8 and pCM7.

Particular named bacterial promoters which can be used include lacI, lacZ, T3, T7, gpt, lambda P_{R}, P_{L} and trp. Eukaryotic promoters include CMV immediate early, HSV thymidine kinase, early and late SV40, adenovirus promoters, LTRs from retrovirus, and mouse metallothionein-I. Selection of the appropriate vector and promoter is well within the level of ordinary skill in the art.

Transcription of the DNA encoding the polypeptides of the present invention by higher eukaryotes can be increased by inserting an enhancer sequence into the expression vector. Enhancers are cis-acting elements of DNA, usually about from 10 to 300 bp that act on a promoter to increase its transcription. Representative examples include the SV40 enhancer on the late side of the replication origin base pairs 100 to 270, a cytomegalovirus early promoter enhancer, the polyoma enhancer on the late side of the replication origin, and adenovirus enhancers.

Generally, recombinant expression vectors also include origins of replication. An expression vector may contain a ribosome binding site for translation initiation, a transcription termination sequence, a polyadenylation site, splice donor and acceptor sites, and/or 5′ flanking or non-transcribed sequences. DNA sequences derived from the SV40 splice and polyadenylation sites may be used to provide required nontranscribed genetic elements. The vector may also include appropriate sequences for amplifying expression. In addition, expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

Large numbers of suitable expression vectors are known to those of skill in the art, and many are commercially available. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, adeno-associated virus, TMV, fowl pox virus, and pseudorabies. However, any other vector may be used as long as it is replicable and viable in a host. Appropriate cloning and expression vectors for use with prokaryotic and eukaryotic hosts are described, e.g., by Sambrook, et al., Molecular Cloning: A Laboratory Manual, Second Edition, Cold Spring Harbor, N.Y., (1989), Wu et al, Methods in Gene Biotechnology (CRC Press, New York, N.Y., 1997), Recombinant Gene Expression Protocols, in Methods in Molecular Biology, Vol. 62, (Tuan, ed., Humana Press, Totowa, N.J., 1997), and Current Protocols in Molecular Biology, (Ausabel et al, Eds.,), John Wiley & Sons, NY (1994-1999). A further discussion of vectors and tissue specific regulatory sequences which are suitable for methods of gene therapy is described herein below.

Appropriate DNA sequences may be inserted into a vector by any of a variety of procedures. In general, the DNA sequence is inserted into an appropriate restriction endonuclease site(s) by standard procedures known in the art. Standard procedures for this and other molecular biology techniques discussed herein are found in many readily available sources, e.g., Sambrook, et al., Molecular Cloning: A Laboratory Manual, Second Edition, Cold Spring Harbor, N.Y., (1989). See also Graham et al. (1988) Virology 63, 614-617 for a rescue recombination technique useful for the construction of, e.g., adenoviral gene delivery vehicles. If desired, a heterologous structural sequence is assembled in an expression vector in appropriate phase with translation initiation and termination sequences, and preferably, a leader sequence capable of directing secretion of translated protein into the periplasmic space or extracellular medium.

The present invention also relates to host cells which are transformed/transfected/transduced with constructs such as those described above, and to progeny of said cells, especially where such cells result in a stable cell line that can be used for assays of eNOS activity, e.g., in order to identify agents which modulate eNOS activity, and/or for production (e.g., preparative production) of the polypeptides of the invention.

As representative examples of appropriate hosts, there may be mentioned: bacterial cells, e.g., E. coli, Streptomyces, Salmonella typhimurium; fungal cells, e.g., yeast; insect cells such as Drosophila S2 and Spodoptera Sf9 (and other insect expression systems); animal cells, including mammalian cells such as CHO, COS (e.g., the COS-7 lines of monkey kidney fibroblasts described by Gluzman, Cell, 23:175 (1981)), C127, 3T3, CHO, HeLa, BHK or Bowes melanoma cell lines; plant cells. The selection of an appropriate host is deemed to be within the knowledge of those skilled in the art based on the teachings herein. Cell lines used for testing putative modulatory agents are commonly mammalian cells whose NO levels are monitored for indications of varying eNOS activity.

Introduction (or delivery) of a construct into a host cell can be accomplished by, e.g., calcium phosphate transfection, DEAE-Dextran mediated transfection, lipofection, a gene gun, or electroporation (Davis, L., Dibner, M., Battey, I., Basic Methods in Molecular Biology, (1986)).

Following transformation of a suitable host strain and growth of the host strain to an appropriate cell density, the selected promoter can be induced by appropriate means (e.g., temperature shift or chemical induction) if desired, and cells cultured for an additional period. The engineered host cells can be cultured in standard nutrient media modified as appropriate for activating promoters (if desired), selecting transformants or amplifying the genes of the present invention. The culture conditions, such as temperature, pH and the like, can be those previously used with the host cell selected for expression, and will be apparent to the ordinarily skilled artisan.

Cells can be typically harvested by centrifugation, disrupted by physical or chemical means, and the resulting crude extract retained for further purification. Alternatively, when a heterologous polypeptide is secreted from the host cell into the culture fluid, supernatants of the culture fluid can be used as a source of the protein. Microbial cells employed in expression of proteins can be disrupted by any standard method, including freeze-thaw cycling, sonication, mechanical disruption, or use of cell lysing agents.

The polypeptide can be recovered and purified from recombinant cell cultures by standard methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography, or the like. Protein refolding steps can be used, as necessary, in completing configuration of the mature protein. High performance liquid chromatography (HPLC) can be employed for final purification steps.

In addition to the methods described above for producing polypeptides recombinantly from a prokaryotic or eukaryotic host, polypeptides of the invention can be prepared from natural sources, or can be prepared by chemical synthetic procedures (e.g., synthetic or semi-synthetic), e.g., with standard peptide synthesizers. Cell-free translation systems can also be employed to produce such proteins using RNAs derived from the DNA constructs of the present invention. Proteins of the invention can also be expressed in, and isolated and/or purified from, transgenic animals or plants. Procedures to make and use such transgenic organisms are standard in the art. Some such procedures are described elsewhere herein.

Of course, recombinant polynucleotides, vectors comprising such polynucleotides, and transfer vehicles (e.g., viral transfer vehicles for use in gene therapy) can also be prepared by standard methods. For example, to prepare adenoviruses, e.g., comprising a recombinant eNOS mutant polynucleotide of the invention, infected cells can be centrifuged and lysed, and the cell lysate can be further treated to isolate (e.g., purify, separate) the viruses from undesirable contaminants, such as cellular components. Among the standard procedures for purifying adenoviruses are, for example, centrifugation or expanded bed adsorption chromatography to remove cell debris and/or to concentrate the virus, size exclusion chromatography, ion exchange (e.g., DEAE) chromatography, ultracentrifugation, ultrafiltration, etc.

The invention disclosed herein also relates to a non-human transgenic animal comprising within its genome one or more copies of the polynucleotides encoding the polypeptides of the invention. The transgenic animals of the invention may contain within their genome multiple copies of the polynucleotides encoding eNOS polypeptide mutants of the invention, or one copy of a gene encoding such polypeptide but wherein said gene is linked to a promoter (e.g., a regulatable promoter) that will direct expression (preferably overexpression) of the eNOS polypeptide mutant within some, or all, of the cells of the transgenic animal. In a preferred embodiment, expression of an eNOS polypeptide mutant of the invention occurs preferentially in vascular tissue. Regulatory sequences, such as tissue specific promoters or enhancers, that can ensure that the eNOS mutants of the invention are expressed preferentially in desired locations, are well known in the art. Some such regulatory elements are discussed elsewhere herein. A variety of non-human transgenic organisms are encompassed by the invention, including e.g., drosophila, C. elegans, zebrafish and yeast. The transgenic animal of the invention is preferably a mammal, e.g., a cow, goat, sheep, rabbit, non-human primate, or rat, most preferably a mouse.

Methods of producing transgenic animals are well within the skill of those in the art, and include, e.g., homologous recombination, mutagenesis (e.g., ENU, Rathkolb et al, Exp. Physiol., 85(6):635-644, 2000), and the tetracycline-regulated gene expression system (see e.g., U.S. Pat. No. 6,242,667; Wu et al, Methods in Gene Biotechnology, CRC 1997, pp.339-366; Jacenko, O., Strategies in Generating Transgenic Animals, in Recombinant Gene Expression Protocols, Vol. 62 of Methods in Molecular Biology, Humana Press, 1997, pp 399-424).

Transgenic organisms are useful, e.g., for providing a source of a polynucleotide or polypeptide of the invention, or for identifying and/or characterizing agents that modulate expression and/or activity of such a polynucleotide or polypeptide. Transgenic animals are also useful as models for disease conditions related to, e.g., expression of a mutant polynucleotide or polypeptide of the invention.

The present invention also relates to a transgenic non-human animal whose genome comprises one or more genes coding for a mutant eNOS disclosed herein in place of the mammalian gene otherwise coding for said polypeptide. Methods of knocking out an eNOS gene and replacing it with a mutant gene are known (see, e.g., Murohara 1998 ibid) for a description of a mouse in which the eNOS gene has been knocked out). Preferably, the transgenic animal is a mouse or rat.

In addition to the methods mentioned above, transgenic animals (or knock out animals into which a transgene is inserted to replace the knocked out gene) can be prepared according to known methods, including, e.g., by pronuclear injection of recombinant genes into pronuclei of one-cell embryos, incorporating an artificial yeast chromosome into embryonic stem cells, gene targeting methods, embryonic stem cell methodology, cloning methods, nuclear transfer methods. See, also, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet., 20:465-499, 1986; Askew et al., Mol. Cell. Bio., 13:4115-4124, 1993; Games et al. Nature, 373:523-527, 1995; Valancius and Smithies, Mol. Cell. Bio., 11:1402-1408,1991; Stacey et al., Mol. Cell. Bio., 14:1009-1016,1994; Hasty et al., Nature, 350:243-246, 1995; Rubinstein et al., Nucl. Acid Res., 21:2613-2617,1993; Cibelli et al., Science, 280:1256-1258, 1998. For guidance on recombinase excision systems, see, e.g., U.S. Pat. Nos. 5,626,159, 5,527,695, and 5,434,066. See also, Orban, P. C., et al., Proc. Natl. Acad. Sci. USA, 89:6861-6865 (1992); O'Gorman, S., et al., Science, 251:1351-1355 (1991); Sauer, B., et al., Polynucleotides Research, 17(1):147-161 (1989); Gagneten, S. et al. (1997) Nucl. Acids Res. 25:3326-3331; Xiao and Weaver (1997) Nucl. Acids Res. 25:2985-2991; Agah, R. et al. (1997) J. Clin. Invest. 100:169-179; Barlow, C. et al. (1997) Nucl. Acids Res. 25:2543-2545; Araki, K. et al. (1997) Nuc. Acids Res. 25:868-872; Mortensen, R. N. et al. (1992) Mol. Cell. Biol. 12:2391-2395 (G418 escalation method); Lakhlani, P. P. et al. (1997) Proc. Natl. Acad. Sci. USA 94:9950-9955 (“hit and run”); Westphal and Leder (1997) Curr. Biol. 7:530-533 (transposon-generated “knock-out” and “knock-in”); Templeton, N. S. et al. (1997) Gene Ther. 4:700-709 (methods for efficient gene targeting, allowing for a high frequency of homologous recombination events, e.g., without selectable markers); PCT International Publication WO 93/22443 (functionally-disrupted).

For generating a transgenic animal, an eNOS polynucleotide of the present invention can be introduced into any non-human animal for generating a transgenic animal, including a non-human mammal, e.g., mouse (e.g., Hogan et al., Manipulating the Mouse Embryo: A Laboratory Manual, Cold Spring Harbor Laboratory, Cold Spring Harbor, N.Y., 1986), pig (e.g., Hammer et al., Nature, 315:343-345, 1985), sheep (e.g., Hammer et al., Nature, 315:343-345, 1985), cattle, rat, or primate (also, e.g., Church, 1987, Trends in Biotech. 5:13-19; Clark et al., Trends in Biotech. 5:20-24, 1987); and DePamphilis et al., BioTechniques, 6:662-680,1988). Transgenic animals can be produced (or propagated) by the methods described in U.S. Pat. No. 5,994,618, and utilized for any of the utilities described therein.

eNOS Polypeptide Binding Partners

The eNOS polypeptide mutants of the present invention, or variants thereof, or cells expressing them, can also be used to assay for specific binding partners, e.g., proteins and nucleic acids that bind specifically to an eNOS polypeptide mutant of the present invention. Such binding partners include, e.g., kinases, phosphatases, and calmodulin. In addition, the eNOS polypeptide mutants of the present invention can be used as immunogens to produce specific antibodies, or antigen-binding fragments, thereto. Standard methods described herein or known in the art can be used to assay for and isolate such specific binding partners of eNOS polypeptide mutants.

By a “specific” antibody or antigen-binding fragment is meant one that binds selectively (preferentially) to an eNOS of the invention, or to a fragment or variant thereof, in particular to a mutated sequence of the invention. An antibody “specific” for a polypeptide means that the antibody recognizes a defined sequence of amino acids within or including the polypeptide.

Antibodies of the invention can be, for example, polyclonal or monoclonal antibodies. The present invention also includes chimeric, recombinant, single chain, and partially or fully humanized antibodies, as well as Fab fragments, or the product of a Fab expression library, and fragments thereof. The antibodies can be IgM, IgG, subtypes, IgG2A, IgG1, etc. Various procedures known in the art may be used for the production of such antibodies and fragments.

Antibodies generated against the polypeptides corresponding to a sequence of the present invention can be obtained, e.g., by direct injection of the polypeptides into an animal or by administering the polypeptides to an animal, e.g., goat, rabbit, mouse, chicken, etc., preferably a non-human. The antibody so obtained will then bind the polypeptide itself. In this manner, even a sequence encoding only a fragment of the polypeptides can be used to generate antibodies binding the whole native polypeptides. Such antibodies can then be used to isolate the polypeptide from tissue expressing that polypeptide. Antibodies can also be generated by administering naked DNA. See, e.g., U.S. Pat. Nos. 5,703,055; 5,589,466; and 5,580,859.

For preparation of monoclonal antibodies, any technique which provides antibodies produced by continuous cell line cultures can be used. Examples include, e.g., the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (e.g., U.S. Pat. No. 4,946,778) can be adapted to produce single chain antibodies to immunogenic polypeptide products of this invention. Also, transgenic animals may be used to express partially or fully humanized antibodies to immunogenic polypeptide products of this invention.

The invention also relates to other specific binding partners which include, e.g., aptamers and PNA.

Diagnostic, Prophylactic, and Therapeutic Uses of eNOS Polypeptide Mutants and Polynucleotides Encoding Such eNOS Polypeptides

Endothelial NO synthases are involved in a variety of functions and activities, e.g. as described herein; and aberrant expression and/or activity of these eNOS polypeptides, and/or aberrant amounts of NO produced by these enzymes, are associated with a variety of disease conditions. Consequently, the eNOS polypeptide mutants and polynucleotides of the present invention, and variants thereof, can be used to modulate eNOS activity in cells to ameliorate such conditions.

In some embodiments, increased expression and/or activity of an eNOS, and a concomitant increase in the production of NO by the eNOS, is associated, e.g., with undesirable angiogenesis, e.g., which allows for undesirable cell proliferation, tumor growth or various neoplastic diseases. Among the conditions associated with increased production of NO are, e.g., various neoplastic diseases (including carcinogenesis, tumoral development and metastases proliferation), resistance of malignant neoplastic tumours to radio or chemotherapy, bladder cancer metastatic or not (cystadenocarcinoma), angiosarcoma, and proliferative retinopathies.

In some embodiments, decreased expression and/or activity of eNOS and concomitant decrease in the amount of NO production by the eNOS, is associated with a variety of conditions, e.g., disease conditions, such as conditions associated with excessive vasoconstriction and/or inadequate vasodilation, e.g., peripheral limb ischemia, peripheral arterial occlusive disease (PAOD) and critical limb ischemia (CLI), atherosclerosis or vascular thrombosis; myocardial ischemia, such as that which results from flow-limiting coronary arterial stenosis; restenosis, e.g., following balloon angioplasty; hypertension, pulmonary hypertension, obstructive airways disease, transplant atherosclerosis, aortic aneurysm, hypercholesterolemia, aging, inflammation, effects of cigarette smoking, congestive heart failure, toxemia of pregnancy, diabetes, diseases of defective angiogenesis, Raynaud's phenomenon, Prinzmetal's angina (coronary vasospasm), cerebral vasospasm, hemolytic-uremia, erectile dysfunction, and poor wound healing.

Other conditions associated with abnormally low amounts of NO include cardiac insufficiency, cardiac decompensation, ischemic cardiomyopathy, dilated or post-transplantation cardiomyopathies, angina pectoris (including instable angina), coronary spasm, post-transplantation coronaropathy, hypercholesterolemia, hyperlipidemia, hypertriglyceridemia, vascular side effects of diabetes mellitus (insulino-dependent or not), vascular side effects of chronic renal insufficiency (uremia), endothelial dysfunction of various origins (atherosclerosis, smoke-addiction, syndrome X, obesity, hypertension, dyslipidemia, resistance to insulin), systemic or auto-immune vasculitis, hyperhomocysteinemia, buerger angeitis, thrombo-embolic disease, deep or superficial vein thrombosis, atherosclerosis with arterial insufficiency in a vascular area (ischemia, including cerebral ischemia or coronary ischemia), pulmonary arterial hypertension, side effects or hemodialysis or peritoneal dialysis.

Insufficient NO is also associated with undesirable contraction of uterine smooth muscle or relaxation of the cervix; thus administration of a mutant eNOS of the invention is useful for, e.g., preventing preterm labor by administration to the uterus, or stimulating or inducing labor by administration to the cervix. Other conditions characterized by insufficient NO include preeclampsia, dysmennorhea and urinary incontinence.

Administration of the eNOS polypeptide mutants of the invention is also useful for modulating the immune response. For example, eNOS polynucleotides of the invention can be introduced into T-cells, platelets, neutrophils, monocytes, or NK cells to regulate their activity. Disease conditions which can benefit by such a procedure include, e.g., atherosclerosis, inflammatory diseases, or autoimmune diseases.

Without wishing to be bound to any particular theory or mechanism, it is proposed that an eNOS polypeptide mutant of the invention is useful in the treatment of ischemic heart disease by promoting both glucose and fatty acid metabolism, as well as by improved nutrient and oxygen supply to the myocytes.

This invention provides methods of screening agents, in vitro or in vivo (e.g., in cell-based assays or in animal models), to identify those agents that modulate synthesis and/or activity of eNOS polypeptides. Such methods can employ the mutant eNOS polypeptides or polynucleotides of the present invention, or variants thereof. Agents that inhibit such synthesis and/or activity (antagonists) may, e.g., result in decreased levels of NO within a patient's cells and resultant physiological alterations resulting therefrom. Agents that enhance such synthesis and/or activity (agonists) may, e.g., result increased levels of NO within the subject cells. For example, the invention relates to a method to identify modulators of eNOS expression, comprising testing putative modulators for their ability to increase or decrease phosphorylation or activity of an eNOS polypeptide mutant of the invention, e.g., as a function of calmodulin and/or calcium concentrations, or to modulate any of the eNOS activities discussed herein. Assay methods to monitor such activities are standard and well known to those of skill in the art.

Agents which inhibit eNOS expression and/or activity can be used to treat, prevent, and/or ameliorate the symptoms of conditions associated with an overexpression or increased activity of an eNOS; and agents which enhance such activity can be used to treat, prevent, and/or ameliorate the symptoms of conditions associated with an underexpression or decreased activity of an eNOS. Inhibitors of eNOS polypeptides (e.g., inhibitors of eNOS activity or expression) can be used, e.g., to treat any of the conditions described elsewhere herein which are associated with an overproduction of, or increased activity of, an eNOS. Stimulators of the eNOS polynucleotides or polypeptide mutants of the present invention can be used, e.g., to treat any of the conditions described elsewhere herein which are associated with an underproduction of, or decreased activity of, an eNOS.

In assaying for potential antagonists or agonists, a variety of functions and/or enzymatic activities which are associated with eNOS can be employed. Typical functions and activities are discussed elsewhere herein. Assays can be performed in vitro, ex vivo or in vivo, and can be performed using any suitable cell or tissue. In vivo assays can be performed using, e.g., transgenic mice as already described, or a humanized mouse in which a human gene coding for a mutant human eNOS disclosed herein is present in place of the mouse gene otherwise coding for such polypeptide mutant.

Any of the assays described herein can, of course, be adapted to any of a variety of high throughput methodologies, as can the generation, identification and characterization of putative inhibitory or stimulatory agents. Agents identified on the basis of their ability to modulate eNOS expression or activity may also be used for modulating other eNOS wild-type or mutant polypeptides, and/or for diagnosing or treating disease conditions associated with one or more eNOS activities.

Potential modulators, e.g., inhibitors or activators, of the invention, include, e.g., small chemical compounds (e.g., inorganic or organic molecules), polypeptides, peptides or peptide polypeptide mutants, polynucleotides, antibodies that bind specifically to the polypeptides of the invention, or the like. Other inhibitory or stimulatory substances may enter cells and bind directly to the DNA neighboring the sequences coding for the polypeptides of the invention, thereby decreasing their expression and thus decreasing intracellular levels of NO, or increasing their expression and thus increasing intracellular levels of NO.

The present invention provides for a means of diagnosing or staging actual or potential diseases or conditions involving altered levels of NO (e.g., which are mediated by or related to eNOS production or activity), by determining the amounts (e.g., the presence or absence, or the quantity) of the eNOS polypeptide mutants of the invention, or their levels of activity, in an animal suspected of having such a disease or condition or being at risk therefore (e.g., “a patient in need of treatment”). For example, the invention provides a process for diagnosing a disease in an animal afflicted therewith, or diagnosing a susceptibility to a disease in an animal at risk thereof, wherein the disease is related, for example, to the presence in a particular cell, tissue or organ of a mutation of the invention, which leads to an undesirable increase or decrease in NO levels in the cell, tissue or organ, preferably wherein the animal is a mammal, more preferably a human. Such diagnostic methods can employ any of the assays described elsewhere herein. For example, one can detect mutated polypeptides using methods based on antibodies or antigen-specific fragments of the invention. Immunological assays include, e.g., ELISA, RIA and FACS assays. When assaying samples for diagnostic purposes, samples may be obtained from any suitable cell, tissue, organ, or bodily fluid from a patient, including but not limited to blood, urine, saliva, tissue biopsy and autopsy material.

The detection of the eNOS polypeptide mutants of the invention can also be useful for research purposes, e.g., when screening cells that have been transfected with plasmids bearing such mutants in order to identify those cells that comprise the mutation.

In accordance with the present invention, an antibody or antigen-binding fragment can be present in a kit, where the kit includes, e.g., one or more antibodies or antigen-binding fragments, a desired buffer, detection compositions, proteins (e.g., eNOS mutants of the invention) to be used as controls.

Assays involving polynucleotides can be used to determine the presence or absence of a mutant eNOS nucleic acid of the invention in a sample and/or to quantify it. Such assays can be used, e.g., for diagnostic, prognostic, research, or forensic purposes. The assays can be, e.g., membrane-based, solution-based, or chip-based.

Any suitable assay format can be used, including, but not limited to, Southern blot analysis, Northern blot analysis, polymerase chain reaction (“PCR”) (e.g., Saiki et al., Science, 241:53, 1988; U.S. Pat. Nos. 4,683,195, 4,683,202, and 6,040,166; PCR Protocols: A Guide to Methods and Applications, Innis et al., eds., Academic Press, New York, 1990), reverse transcriptase polymerase chain reaction (“RT-PCR”), anchored PCR, rapid amplification of cDNA ends (“RACE”) (e.g., Schaefer in Gene Cloning and Analysis: Current Innovations, Pages 99-115,1997), ligase chain reaction (“LCR”) (EP 320 308), one-sided PCR (Ohara et al., Proc. Natl. Acad. Sci., 86:5673-5677,1989), indexing methods (e.g., U.S. Pat. No. 5,508,169), in situ hybridization, differential display (e.g., Liang et al., Nucl. Acid. Res., 21:3269-3275,1993; U.S. Pat. Nos. 5,262,311, 5,599,672 and 5,965,409; WO97/18454; Prashar and Weissman, Proc. Natl. Acad. Sci., 93:659-663, and U.S. Pat. Nos. 6,010,850 and 5,712,126; Welsh et al., Nucleic Acid Res., 20:4965-4970,1992, and U.S. Pat. No. 5,487,985) and other RNA fingerprinting techniques, nucleic acid sequence based amplification (“NASBA”) and other transcription based amplification systems (e.g., U.S. Pat. Nos. 5,409,818 and 5,554,527; WO 88/10315), polynucleotide arrays (e.g., U.S. Pat. Nos. 5,143,854, 5,424,186; 5,700,637, 5,874,219, and 6,054,270; PCT WO 92/10092; PCT WO 90/15070), QBeta Replicase (PCT/US87/00880), Strand Displacement Amplification (“SDA”), Repair Chain Reaction (“RCR”), nuclease protection assays, subtraction-based methods, or Rapid-Scan™.

Additional useful methods include, but are not limited to, e.g., template-based amplification methods, competitive PCR (e.g., U.S. Pat. No. 5,747,251), redox-based assays (e.g., U.S. Pat. No. 5,871,918), Taqman-based assays (e.g., Holland et al., Proc. Natl. Acad, Sci., 88:7276-7280,1991; U.S. Pat. Nos. 5,210,015 and 5,994,063), real-time fluorescence-based monitoring (e.g., U.S. Pat. No. 5,928,907), molecular energy transfer labels (e.g., U.S. Pat. Nos. 5,348,853, 5,532,129,5,565,322, 6,030,787, and 6,117,635; Tyagi and Kramer, Nature Biotech., 14:303-309,1996). Any method suitable for single cell analysis of gene or protein expression can be used, including in situ hybridization, immunocytochemistry, MACS, FACS, flow cytometry, etc. For single cell assays, expression products can be measured using antibodies, PCR, or other types of nucleic acid amplification (e.g., Brady et al., Methods Mol. & Cell. Biol. 2, 17-25, 1990; Eberwine et al., 1992, Proc. Natl. Acad. Sci., 89, 3010-3014, 1992; U.S. Pat. No. 5,723,290). These and other methods can be carried out conventionally, e.g., as described in the cited references.

The invention also provides methods for diagnosing a disease in an animal afflicted therewith, or diagnosing susceptibility to a disease in an animal at risk thereof, wherein the disease is associated, for example, to expression of a polynucleotide encoding an eNOS polypeptide, comprising determining the amount of the polynucleotide in a cell from the animal, wherein the animal is preferably a mammal and most preferably a human. Any of the assay methods described herein, or otherwise known in the art, can be used to determine the presence of and/or to quantitate, such polynucleotides.

A polynucleotide sequence coding for part or all of a eNOS polypeptide mutant of the invention may act as a reference for the development of probes, e.g., as long as 30 to 45 nucleotides, or longer, that can be used e.g., to probe the genome of animals suspected of being at risk for disease, or having such disease, or for detecting the presence of such a mutant polynucleotide for research purposes, e.g., when screening cells that have been transtected with plasmids bearing such mutants in order to identify those cells that comprise the mutation.

A hybridization probe of this type preferably has at least 7 or 8 bases, more preferably about 10, 11, 12, 13, 14 or 15 bases, and most preferably at least about 30 bases, and exhibits about 65-100% sequence identity to part or all of the sequence coding for an eNOS polypeptide mutant of the invention. Hybridization probes are specific to, or for, a selected polynucleotide. The phrases “specific for” or “specific to” a polynucleotide have a functional meaning that the probe can be used to identify the presence of one or more target genes or polynucleotide sequences in a sample. The probe is specific in the sense that it can be used to detect a polynucleotide above background noise (“non-specific binding”).

In accordance with the present invention, a polynucleotide can be present in a kit, where the kit includes, e.g., one or more polynucleotides (such as a hybridization probe), a desired buffer (e.g., phosphate, Tris, etc.), detection compositions, RNA or cDNA to be used as controls (e.g., comprising a mutant of the invention), libraries, etc. The polynucleotide can be labeled or unlabeled, with radioactive or non-radioactive labels as known in the art.

The invention is also related to therapeutic or prophylactic methods of combating eNOS mediated or associated with diseases or conditions described herein, e.g., by administering an agent that affects production and/or activity of an eNOS, or by administering an agent such as a mutant eNOS polypeptide or polynucleotide of the invention. Such treatment can inhibit ameliorate such diseases or conditions.

Such agents can be administered to patients in need thereof by standard procedures. Suitable routes of delivery are well known to those of skill in the art and include, but are not limited to, intravascular, intramuscular, intraperitoneal, intradermal, intraarterial and oral methods.

Such agents can be formulated into pharmaceutical compositions comprising pharmaceutically acceptable excipients, carriers, etc., using standard methodologies. Formulations and excipients which enhance transfer (promote penetration) of an agent across cell membranes or which protect against degradation are also well-known in the art. For example, a delivery vehicle (for in vivo or ex vivo transfer, e.g., of a polynucleotide) may be delivered to a target cell by any of a variety of standard procedures, including, e.g., liposome mediated transfection, e.g., in which the liposomes are cationic liposomes containing cholesterol derivatives such as SF-chol or DC-chol; transfection with lipofectamine, or the like. Typical methods are described, e.g., in U.S. Pat. No. 5,656,565; Mannino et al. (1988) BioTechniques 6, 682-690 and references therein; and Gao et al. (1991) Biochem Biophys Res Comm 179, 280-285.

In one embodiment, agents, which are administered to a patient suffering from a condition associated with eNOS activity, are administered locally to the site at which the disease condition is expressed. Such local delivery can avoid unwanted effects (e.g., side effects) resulting from, e.g., induction of NO in a non-disease related cell or tissue.

For example, molecules can be delivered directly to heart or skeletal muscle, including cardiac myocytes and skeletal myocytes. Polypeptides or polynucleotides of the invention can be delivered to the myocardium by direct intracoronary (or graft vessel) injection using standard percutaneous catheter based methods under fluoroscopic guidance, e.g., at an amount sufficient for a transgene to be expressed to a degree which allows for highly effective therapy. The injection may be made deeply into the lumen (e.g., about 1 cm within the arterial lumen) of the coronary arteries (or graft vessel), and preferably be made in both coronary arteries, as the growth of collateral blood vessels is highly variable within individual patients. By injecting the material directly into the lumen of the coronary artery by coronary catheters, it is possible to target the gene effectively, and to minimize loss of the recombinant vectors or polypeptide to the proximal aorta during injection. Gene expression when delivered in this manner does not occur in hepatocytes and viral RNA cannot be found in the urine at any time after intracoronary injection. Any variety of coronary catheter, or a Stack perfusion catheter, for example, can be used in the present invention. In addition, other techniques known to those having ordinary skill in the art can be used for transfer of a mutant eNOS of the invention to the arterial wall. See also Giordano et. al., (1994) Clin Res 42, 123A.

For treatment of peripheral vascular disease, a disease characterized by insufficient blood supply to the legs, a polynucleotide of the invention may be delivered by a catheter inserted into the proximal portion of the femoral artery or arteries, thereby effecting transfer into the cells of the skeletal muscles receiving blood flow from the femoral arteries. See, e.g., U.S. Pat. No. 5,792,453.

Agents of the invention may also be transferred directly to the brain or spinal cord, to the uterus or cervix (e.g., in creams or suppositories), or to any desirable location, using standard procedures.

In another embodiment, therapeutic molecules (e.g., mutant polypeptides or polynucleotides of the invention) are administered systemically, but are modified so that they are targeted to a cell, tissue or organ of interest, using standard methods. For example, polynucleotides can be placed under the control of tissue-specific regulatory elements, such as promoters or enhancer elements.

By fusing, for example, tissue-specific transcriptional control sequences of left ventricular myosin light chain-2 (MLC[2V]) or myosin heavy chain (MHC) to a transgene such as the eNOS genes of the invention within a construct, such as an adenoviral construct, transgene expression is limited to ventricular cardiac myocytes. The efficacy of gene expression and degree of specificity provided by MLC[2V] and MHC promoters with lacZ have been determined, using a recombinant adenoviral system. Cardiac-specific expression has been reported by Lee et al. (J. Biol. Chem. 267:15875-15885 (1992)). The MLC[2V] promoter is comprised of 250 bp, and fits easily within, e.g., adenoviral-5 packaging constraints. The myosin heavy chain promoter, known to be a vigorous promoter of transcription, provides a reasonable alternative cardiac-specific promoter and is comprised of less than 300 bp. Skeletal muscle specific promoters can also be used (see e.g., Hauser et al.(2000) Mol. Therapy 2:16-26; Li et al. (1999) Nature Biotech. 17:241-245; and Patent WO 99/02737. Smooth muscle cell promoters such as SM22 alpha promoter (Kemp et al., (1995) Biochem J 310 (Pt3):1037-43) and SM alpha actin promoter (Shimizu et al. (1995) J Biol Chem 270(13):7631-43) are also available. By using such a tissue specific promoter and delivering a transgene in vivo, it is believed that, e.g., the cardiac myocyte alone (that is without concomitant expression in endothelial cells, smooth muscle cells, and fibroblasts within the heart) will provide adequate expression of the eNOS polypeptide mutants of the present invention. Limiting expression to the cardiac myocyte or skeletal muscle also has advantages regarding the utility of gene transfer for the treatment of clinical myocardinal ischemia or peripheral ischemia. By limiting expression to the heart or skeletal muscle, one avoids the potentially harmful effect of angiogenesis in tissues such as the retina. In addition, of the cells in the heart, the myocyte would likely provide the longest transgene expression since the cells do not undergo rapid turnover; expression would not therefore be decreased by cell division and death as would occur with endothelial cells.

Endothelial-specific promoters are already available for this, or other, purposes. Examples of endothelial specific promoters include the Tie-2 promoter (Schlaeger et al. (1997) Proc Natl Acad Sci 1;94(7):3058-63), the endothelin promoter (Lee et al. (1990) J. Biol. Chem. 265:10446-10450), and the eNOS promoter (Zhang et al. (1995) J. Biol. Chem 270(25):15320-6) and (Bu and Quertermous (1997) J. Biol. Chem. 272:32613-32622).

In one embodiment of the invention, an eNOS polypeptide mutant, or variant thereof, is administered to a patient in need of such therapy, and such a polypeptide can, e.g., compensate for reduced or aberrant expression or activity of an eNOS, including e.g., abnormally low levels of NO in a cell, tissue, or organ of the patient. In another embodiment, the methods of the invention relate to a method of stimulating collateral vessel development in ischemic diseases with deficient endogenous angiogenesis, specifically, peripheral vascular disease, myocardial ischemia or critical limb ischemia (CLI) in a patient, comprising delivering an eNOS polypeptide mutant of the invention.

In a preferred embodiment, the eNOS polypeptide mutant is modified in order to enhance its ability to enter a cell. For example, a fusion polypeptide YGRKKRRQRRR (also called the protein transduction domain, PTD) of the HIV-TAT polypeptide at the N-terminus of a polypeptide of interest can be made in E. coli and the denatured form of the protein purified. This fusion polypeptide (e.g., PTD-eNOS) can be introduced into a patient for therapy. Methods for transducing denatured full-length proteins into cells has been described in the art (see e.g., Nature Medicine (1998) Vol. 4(12):1449).

In another embodiment, the invention relates to a method of treating a condition characterized by cells or tissues having an abnormally low activity or amount of eNOS, comprising delivering a polynucleotide encoding a mutant eNOS polypeptide of the invention, i.e., a method of gene therapy, in which a polynucleotide of the invention is delivered in a gene delivery vehicle. Such methods can be used to treat any of the conditions described elsewhere herein. For example, the invention relates to a method to stimulate collateral vessel development in ischemic diseases with deficient endogenous angiogenesis, specifically peripheral vascular disease and/or myocardial ischemia in a patient comprising delivering a transgene coding for a mutant eNOS polypeptide of the invention.

The gene delivery vehicle may be of viral or non-viral origin (see generally, Jolly, Cancer Gene Therapy 1:51-64 (1994) Kimura, Human Gene Therapy 5:845-852 (1994); Connelly, Human Gene Therapy 1:185-193(1995); and Kaplitt, Nature Genetics 6:148-153(1994). Gene therapy vehicles for delivery of constructs including, e.g., a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651, EP 0 345 242 and WO 91/02805.

Packaging cell lines suitable for use with the above-described retroviral vector constructs may be readily prepared (see, e.g., PCT publications WO 95/30763 and WO 92/05266), and used to create producer cell lines (also termed vector cell lines) for the production of recombinant vector particles. Within preferred embodiments of the invention, packaging cell lines are made from human (such as HT1080 cells) or mink parent cell lines, thereby allowing production of recombinant retroviruses that can survive inactivation in human serum.

The methods of the present invention can also employ alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250 ATCC VR-1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et al., P.N.A.S. 90:10613-10617 (1993).

In a preferred embodiment, adenoviral vectors are used. A variety of modified adenoviral vectors (e.g., of Ad5 or Ad2), particularly non-replicative vectors and/or helper independent viruses, are well-known in the art. Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Eisler et al., P.N.A.S. 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5: 1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992), may be employed.

Other gene delivery vehicles and methods may be employed, including polycationic condensed DNA linked or unlinked to killed adenovirus alone, for example, Curiel, Hum. Gene Ther. 3:147-154 (1992); ligand-linked DNA, for example, see Wu, J. Biol. Chem. 264:16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994) and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697 and WO 91/14445, and EP No. 0 524 968.

Further non-viral delivery suitable for use includes mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other standard methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

Gene therapy methods of delivering polynucleotides to patients in need of treatment can be performed in vivo (administering the polynucleotide directly to the patient) or ex vivo (cell-based therapy which involves introducing the polynucleotide to a cell, e.g., a cell taken from the patient to be treated, or a cell which is not from the patient to be treated, and then introducing the transfected cell to the patient.)

Mutant eNOS polypeptides or polynucleotides of the invention can be administered alone or in combination with other agents, e.g., angiogenic factors including, but not limited to, FGF, HGF, VEGF and bFGF, especially VEGF or bFGF, before, during or after the administration of the eNOS. Methods to prepare, administer, and test the effects of administration of, such growth factors are standard. See, e.g., Papapetropoulos et al., (1997) J Clin Invest 100, 3131-3139, Brock et al., (1991) Am J Pathol 138, 213-221, and Ku et al., (1993) Am J. Physiol. 265, H 586-592, and references therein. Such growth factors can be cloned into appropriate expression vectors (either independently or into a vector which expresses an eNOS polynucleotide of the invention), using standard procedures. See, e.g., Rivard et al., (1999) Am J Pathol 154, 355-363 for a method to induce angiogenesis by intramuscular gene therapy with VEGF.

In the foregoing and in the following examples, all temperatures are set forth uncorrected in degrees Celsius; and, unless otherwise indicated, all parts and percentages are by weight.

The following samples are offered by way of illustration and are not intended to limit the invention in any way.

### EXAMPLES

### Example 1

### eNOS Polypeptide Mutants and Recombinant Plasmid and Viral Vectors

Plasmid vectors encoding eNOS polypeptides having a single or double were generated for plasmid vector delivery and expression of eNOS wild-type and polypeptide mutants in cells in vitro and in vivo. The mutants were generated using Kunkel site-directed mutagenesis directly in the eNOS polynucleotide sequence (Kunkel, T. A. PNAS 1985; 82:488-492). The mutations were confirmed by sequencing. The cDNAs of the wild-type mutant constructs were cloned into the plasmid vector, pShuttle-CMV, placing the polynucleotide encoding the eNOS polypeptide within a CMV expression cassette. Consequently, in these constructs the polynucleotide was operably linked to a CMV promoter such that the promoter drove the expression of the encoded eNOS polypeptide mutant in cells.

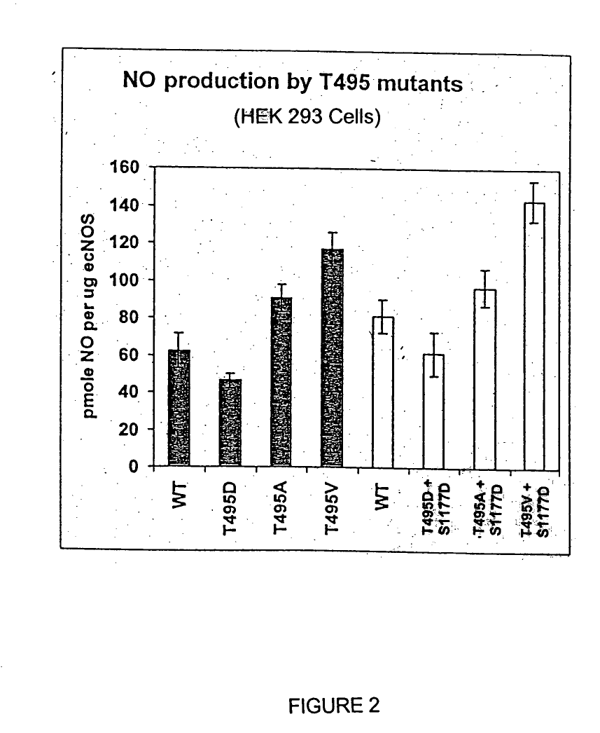

In the eNOS polypeptide mutants having a single amino acid substitution, the Thr corresponding to position 495 in the calmodulin-binding site of human eNOS (see FIG. 1) was substituted to an Ala, Asp, or Val (designated mutants T495A, T495D, T495V, respectively). In the eNOS polypeptide mutants having a double amino acid substitution, the Ser corresponding to position 1177 was substituted to Asp and, additionally, the Thr corresponding to position 495 was substituted to Ala, Asp, or Val (designated T495A+S1177D, T495D+S1177D, T495V+S1177D, respectively). These mutations were confirmed by sequencing and tested for NO production in HEK 293 cells (Example 2 and FIG. 2)

Adenovirus vectors encoding eNOS polypeptides having the single and double mutations described above, were generated according to a method described by He et al (1998) PNAS 95(5), 2509-2514, and used for viral vector delivery of eNOS wild-type and polypeptide mutants in cells in vitro and in vivo. The pShuttle vectors carrying the polynucleotides encoding an eNOS polypeptide mutant (as described above) were co-transformed into E. coli. BJ5183, along with a plasmid containing an E1 and E3-deleted Ad5 genome. The adenovirus vector backbone was derived from Adenovirus 5. In this vector backbone, the E1 region of the Adenoviral sequence is deleted between nucleotide 454 and 3333, and a partial E3 deletion (nucleotides 30004 to 30750) is replaced with 645 bp foreign DNA. A polynucleotide can be inserted at the site of the E1-deletion such that the CMV promoter (at −632 to +7) and the SV40 polyadenylation signal are operably linked to the polynucleotide for expression of a polypeptide encoded by the polynucleotide.

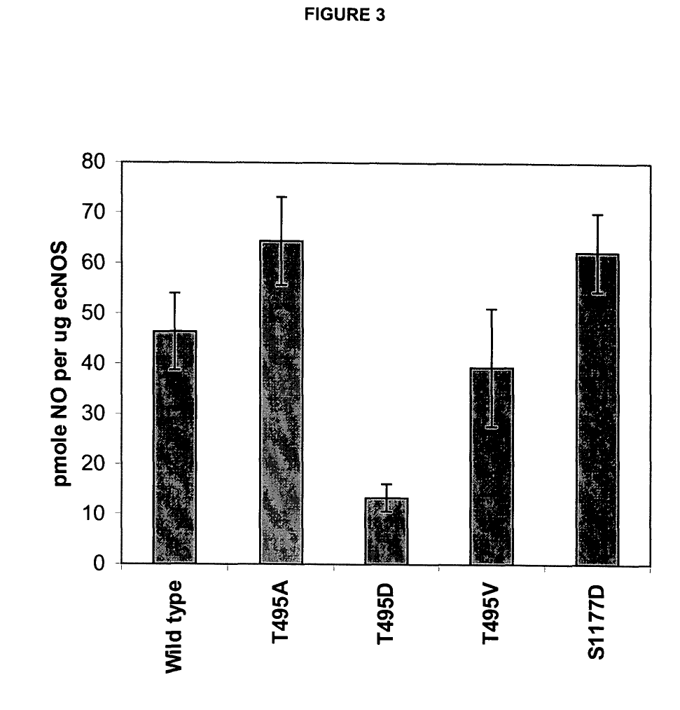

The resulting recombinant adenovirus plasmids encoding an eNOS polypeptide mutant were then selected and confirmed by restriction endonuclease analyses. The corresponding viruses were rescued by transfection of 293 cells with the recombinant adenovirus genomes excised from the plasmids and the viruses were then amplified in 293 cells, purified by standard CsCl gradient purification, and used for testing for NO production in HAEC (Example 3 and FIG. 3).

Additionally, the eNOS polypeptide mutant NOS1177D (provided by Sessa et al., Yale University) has an amino acid substitution to Asp at a position corresponding to amino acid residue 1177 in the reductase domain of SEQ ID NO: 1. In order to test the activity of this eNOS polypeptide mutant in cells, a polynucleotide encoding this mutant wax inserted into the adenovirus backbone (as described above in Example 1) at the position where the E1 position is deleted. The resulting recombinant vector, Ad5NOS1177D, encodes the eNOS polypeptide mutant NOS1177D. The recombinant vector Ad5NOS1177D was transfected into packaging cells and the resulting virus were plaque purified, and subjected to two rounds of amplification. Virus from the second amplification were used to inoculate a large-scale infection of HEK293 cells in a 3L-bioreactor. The resulting virus were then purified by two rounds of CsCl gradient separation and dialyzed against 10 mM Tris pH 8.0, 2 mM MgCl₂ and 4% sucrose. Aliquots of the purified recombinant virus were also used for testing NO production in HAEC (see Examples 3, 5, and 7).

Ad5EGFP is a control and is an adenovirus vector encoding the reporter gene, green fluorescent protein (GFP), and was prepared by Collateral Therapeutics, then amplified in HEK293 cells, and purified by FPLC. The purified virus was then dialyzed against PBS pH7.2 and 2% sucrose.

Aliquots of the purified control virus were stored at −80° C. for use as a control in subsequent experiments (see Examples 5 and 7).

### Example 2

### Detection and Measurement of the Activity of eNOS Polypeptide Mutants in HEK 293 Cells

In order to test and measure the activity of eNOS polypeptide mutants in HEK 293 cells, plasmid vectors encoding an eNOS polypeptide mutant (described above in Example 1) were used to deliver and express the polypeptide mutants in HEK 293 cells. The HEK 293 cells were first plated in 6-well plates, in 2 ml per well of Growth Medium (Alpha MEM (Gibco 12561-056), containing 10% FBS (SeraCare), 2 mM additional L-glutamine and 50 μg/ml gentamicin. When the cells were about 75% confluent, they were transfected with a plasmid shuttle vector encoding the T495A, Thr495D, or T495V eNOS polypeptide mutant (as described above in Example 1), or a wild-type human (WT) eNOS (SEQ ID NO: 1), or both.

The transfection was performed by mixing 8 μg the plasmid shuttle vector encoding WT eNOS or a mutant eNOS, 60 μl Lipofectamine 2000 (Invitrogen) and 200 I OptiMEM (Gibco), and after incubating 30 minutes at room temperature, adding 111 μl of the mixture plus 420 μl OptiMEM to each well containing HEK 293 cells. After incubation at 37° C. for 2.5 hours, 2 ml of Growth Medium was added to each well.

After two days (cells incubated at 37° C., 5% CO₂), NO production by the cells was measured using chemiluminescence, after which the cells were lysed and the lysates assayed for eNOS protein content using an ELISA assay as described below. NO production was normalized to the amount of eNOS protein, in order to correct for variations in transfection efficiency between the different plasmids.

Measurement of NO Production.

The medium was removed, and each well was washed twice with 2 ml NO Analyzer Buffer (5 mM Na HEPES, 140 mM NaCl, 5 mM KCl, 1 mM MgCl₂, 10 mM glucose, 10 mM CaCl₂, 5 mM L-arginine, pH 7.5). Then the buffer was replaced with 1 ml of NO Analyzer Buffer containing 100 U/ml superoxide dismutase and 40 ng/ml VEGF. The wells were covered with parafilm, and after incubation for 30 minutes at 37° C., 0.8 ml of the buffer above the cells was injected into a Siemens NOA280 chemiluminesence detector for measurement of NO according to the manufacturer's instructions. Authentic NO gas was used as a standard. After NO measurements were completed, the remaining buffer on the cells was removed, and the cells were lysed in 0.6 ml Lysis Buffer (0.5% NP-40, 50 mM Tris-HCl pH 7.5, 1 μg/ml pepstatin A, 1 μg/ml leupeptin, 5 μg/ml aprotinin, 24 μg/ml Pefabloc SC (Boehringer Mannheim)) and stored at −20° C.

Measurement of eNOS Protein:

96-well ELISA plates (Costar 3590) were coated with 100 μl per well of Coating Antibody (rabbit polyclonal anti-eNOS), 5 μg/ml in 50 mM Na carbonate buffer, pH 9.5 and incubated overnight at 4° C. The polyclonal antibody (Babco) was collected from rabbits immunized with a peptide corresponding to residues 599 to 614 of human eNOS coupled to keyhole limpet hemocyanin, and purified using Protein G Sepharose (Amersham). Plates were blocked with 200 μl/well of 0.5% I-Block (Tropix) in PBS+0.01% Tween 20 and incubated overnight at 4° C. Plates were then washed three times with 350 μl per well PBS+0.5 ml/L Tween 20. HEK 293 cell lysates containing eNOS were added to the plate, diluted five- or ten-fold into a final volume of 60 μl/well with Lysis Buffer, and incubated 1.5 to 2 hours at room temperature. Plates were then washed three times with 350 μl per well PBS+0.5 ml/L Tween 20.

The detection antibody, a monoclonal anti-eNOS antibody (Transduction Labs N30020) which was europium-labeled as described in Ref. 2, was added as follows: 125 ng/ml europium-labeled antibody in Wallac Assay Buffer (Wallac/PerkinElmer 1244-111), 100 μl/well. Plates were incubated 1.5 hours at room temperature. Plates were then washed three times with 350 μl per well PBS+0.5 ml/L Tween 20. Wallac Enhancement Solution (Wallac/PerkinElmer 1244-105) was then added, 100 μl/well. Plates were covered with plate sealers and stored overnight at 4° C., and then, after mixing for 10 minutes, plates were read in a Wallac 1420 VICTOR² multilabel counter (PerkinElmer Life Sciences), monitoring time-resolved fluorescence at 615 nm. (Aberle S. et al., Nitric Oxide 1, 226 (1997); Meurer J et al., Methods in Enzymology 359, 433-444 (2002).

The results indicate that the eNOS polypeptide mutants stimulated the production of NO in HEK 293 cells, and that the single mutants, T495A and T495V, and double mutants, T495A+S 1177D and T495A+S1177D, stimulated an increased level of NO production as compared to wild-type eNOS (FIG. 2).

### Example 3

### Detection and Measurement of the Activity of eNOS Polypeptide Mutants in HAE Cells

350,000 human aortic endothelial cells (HAEC) per well were plated in 6 well plates in 4 ml of EGM growth medium (Cambrex) containing 10% FBS. Cells were cultured at 37° C. in 5% CO₂. The next day adenovirus (2×10⁹ total viral particles per well, approximately 2×10⁷ infectious particles per well) encoding wild-type or mutant eNOS (Thr495Ala, Thr495Asp, Thr495Val or Ser 177Asp) was added to each well. After 4 hours of incubation with the virus, the medium was removed and replaced with 2 ml of EBM growth medium (Cambrex) supplemented with 0.1% gelatin and 30 μM sepiapterin (Sigma). After 20 hours, NO production by the cells was measured using chemiluminescence, after which the cells were lysed and the lysates assayed for eNOS protein content using ELISA as described below. NO production was normalized to the amount of eNOS protein, in order to correct for variations in expression level as a result of differences in transfection efficiency with the different adenovirus constructs carrying the different eNOS mutants.

The results indicate that the eNOS polypeptide mutants stimulated the production of NO in HEK 293 cells, and that the single mutants, T495A and T495D stimulated an increased level of NO production as compared to wild-type eNOS (FIG. 3). The results of this study differs from those described in the Example 2, in that the cells were stimulated with VEGF in order to stimulate NO release, whereas as a calcium ionophore was used in the study described in Example 2. In addition, the adenovirus infection produced more eNOS protein per cell (and more NO) than transfection with plasmid DNA. Consequently, over expression of eNOS in this study may have contributed to the level of NO activity observed for the eNOS polypeptide mutants.

Human aortic endothelial cells contain endogenous wild-type eNOS, but the amount of NO produced from the over-expressed mutant eNOS is approximately 20 times that of the endogenous eNOS. In the data described in Examples 2 and 3, the NO production is normalized to the amount of eNOS protein, so the eNOS polypeptide mutants have activities in the same range. It is possible that the differences between the level of detected NO production by the different eNOS polypeptide mutants, using adenoviral vectors and plasmid vectors and different cell types, is due to other limiting factors in the cell, such as cofactor availability.

Thus, further testing of eNOS polypeptide mutants in HAEC can further elucidate the effects of eNOS expression and activity in different cell types.

### Example 4

### Western Blots of Mouse Skeletal Muscle Lysate for Determination of Different eNOS Isoforms

Different isoforms of eNOS can be detected using Western Blot analysis and isolated by standard methods known in the art. In this Example, mouse skeletal muscle lysate was used to detect different murine eNOS isoforms.

SDS-PAGE:

For each sample, 30 μl of muscle homogenate was added to 10 μl 4× sample buffer (Invitrogen Cat. No. NP0007) containing 100 mM dithiothreitol. After heating for 7 to 8 minutes at 100° C., each sample was loaded onto a 10% Tris-glycine SDS-PAGE gel (BMA PAGEr Cat. No. 59102). Where needed, 1 μl of HEK cell lysate (in 40 μl of 1× sample buffer) containing recombinant human eNOS was added as a positive control. Prestained protein markers (10 μl of Invitrogen LC5725) were also loaded in one lane of the gel. Gel was run for 1.5 hours at 130V (constant voltage) in 1× Laemmli Running Buffer provided by the Berlex media prep department.

Blotting onto Nitrocellulose:

Proteins were transferred onto nitrocellulose for 2 hours at 20V (constant) using Novex/Invitrogen apparatus (apparatus was presoaked in transfer buffer). (Transfer buffer=100 ml 10× Transfer buffer, provided by Berlex media prep+200 ml methanol+700 ml H₂O.) After blotting, nitrocellulose was stored overnight at 4° C. in 20 ml TBS+5% nonfat dry milk. (TBS=0.02 M Tris HCl, 0.12 M NaCl, pH 7.5).

Detection of Proteins Using Antibodies (All Steps at Room Temperature):

Blots were incubated in a first antibody (anti-eNOS or anti-bNOS mouse monoclonal BD/Transduction Labs diluted 1:2000 in TBS+0.1% Tween 20+5% nonfat dry milk) for 1 hr 15 min. Blots were then washed as follows: one 10-min and two 5-min washes in TBS+0.1% Tween 20. Blots were then incubated in a second antibody (peroxidase-conjugated goat anti-mouse IgG, Chemicon Intl. AP308P or Roche 1814168, diluted 1:3000 in TBS+0.1% Tween 20+5% nonfat dry milk) for 1 hr. After washing as described above, plus an additional 5-min wash in TBS (no Tween), blots were incubated for 1 min in ECL reagent (Amersham Pharmacia RPN2106). Then blots were covered with Saran Wrap and exposed against Amersham Pharmacia Hyperfilm ECL (RPN 1674A) for 1 to 5 minutes and the film was developed.

In the claims:

1. An isolated eNOS polypeptide mutant having one or more mutations in an amino acid sequence corresponding to a functional domain of a mammalian eNOS, wherein at least one of said mutations is:
i) at a position corresponding to an amino acid residue in a calmodulin-binding domain that is phosphorylated in mammalian cells; and
ii) not an amino acid substitution to Ala or Asp, wherein said eNOS polypeptide mutant has one mutation and said one mutation is at said position.

2. The eNOS polypeptide mutant according to claim 1, wherein said amino acid residue is amino acid residue 495 of a human eNOS.

3. The eNOS polypeptide mutant according to claim 2, wherein the amino acid sequence of said human eNOS is SEQ ID NO: 1.

4. The eNOS polypeptide mutant according to claim 3, wherein said mutation at a position corresponding to amino acid residue 495 is an amino acid substitution to Gly, Val, Leu, Ile, Pro, Phe, Tyr, Trp, Met, Ser, Cys, Glu, Asn, Gln, Lys, Arg, or His.

5. The eNOS polypeptide mutant according to claim 4, wherein said mutation at a position corresponding to amino acid residue 495 is an amino acid substitution to Val, Leu, or Ile.

6. The eNOS polypeptide mutant according to claim 5, wherein said mutation at a position corresponding to amino acid residue 495 is an amino acid substitution to Val.

7. The eNOS polypeptide mutant according to claim 3, wherein said polypeptide mutant further comprises at least one mutation in one or more functional domains other than said calmodulin-binding domain.

8. The eNOS polypeptide mutant according to claim 7, wherein said mutation at a position corresponding to amino acid residue 495 is an amino acid substitution to Ala, Val, Leu, or Ile.

9. The eNOS polypeptide mutant according to claim 8, wherein said mutation at a position corresponding to amino acid residue 495 is an amino acid substitution to Ala or Val.

10. The eNOS polypeptide mutant according to claim 9, wherein said mutation at a position corresponding to amino acid residue 495 is an amino acid substitution to Val.

11. The eNOS polypeptide mutant according to claim 8, wherein said polypeptide mutant further comprises at least one mutation in an amino acid sequence corresponding to a myristolyation site of said human eNOS.

12. The eNOS polypeptide mutant according to claim 11, wherein said polypeptide mutant comprises a mutation at a position corresponding to amino acid residue 2 of said human eNOS.

13. The eNOS polypeptide mutant according to claim 12, wherein said mutation at a position corresponding to amino acid residue 2 is an amino acid substitution to Ala.

14. The eNOS polypeptide mutant according to claim 8, wherein said polypeptide mutant further comprises at least one mutation in an amino acid sequence corresponding to a reductase site of said human eNOS.

15. The eNOS polypeptide mutant according to claim 14, wherein said polypeptide mutant comprises a mutation at a position corresponding to amino acid residue 1177 of said human eNOS.

16. The eNOS polypeptide mutant according to claim 15, wherein said mutation at a position corresponding to amino acid residue 1177 of said human eNOS is an amino acid substitution to Asp.

17. The eNOS polypeptide mutant according to claim 16, wherein said polypeptide mutant further comprises a mutation at a position corresponding to amino acid residue 2 of said human eNOS and said mutation is an amino acid substitution to Ala.

18. The eNOS polypeptide mutant according to claim 1, wherein the phosphorylation of said polypeptide mutant is increased or decreased, as compared to a reference eNOS polypeptide.

19. The eNOS polypeptide mutant according to claim 1, wherein said polypeptide has an increased binding affinity for calmodulin, as compared to a reference eNOS polypeptide.

20. The eNOS polypeptide mutant according to claim 1, wherein Ca++ dependence is decreased in Ca++-calmodulin mediated stimulation of said polypeptide as compared to a reference eNOS polypeptide.

21. The eNOS polypeptide mutant according to claim 1, wherein said polypeptide mutant has increased eNOS activity, as compared to a reference eNOS polypeptide.

22. The eNOS polypeptide mutant according to claim 21, wherein said activity is the generation of NO.

23. The eNOS polypeptide mutant according to claim 21, wherein said activity is reductase activity.

24. The eNOS polypeptide mutant according to claim 18, 19, 20, 21, 22, or 23 wherein the amino acid sequence of said reference polypeptide is, or is derived from, the amino acid sequence of a human eNOS.

25. The eNOS polypeptide mutant according to claim 24, wherein the amino acid sequence of said reference polypeptide is, or is derived from, SEQ ID NO: 1.

26. An isolated eNOS polypeptide mutant, wherein the amino acid sequence of said polypeptide mutant is substantially homologous to the amino acid sequence of the eNOS polypeptide mutant of claim 1.

27. An isolated eNOS polypeptide mutant, wherein the amino acid sequence of said polypeptide mutant has a 95-99% sequence identity to the amino acid sequence of said polypeptide mutant of claim 26.

29. An isolated polynucleotide encoding the polypeptide mutant of claim 1.

30. A recombinant vector comprising the polynucleotide of claim 29 operably linked to at least one regulatory sequence.

31. A pharmaceutical composition comprising the polypeptide mutant of claim 1.

32. A pharmaceutical composition comprising the polynucleotide of claim 29.

33. A binding partner of the polypeptide mutant of claim 1.

34. The binding partner according to claim 33, wherein said binding partner is a polypeptide.

35. The binding partner according to claim 34, wherein said binding partner is an antibody or antigen-specific antibody fragment.

36. A method of modulating eNOS activity in a cell, comprising administering to said cell the polypeptide mutant of claim 1.

37. A method of modulating eNOS activity in a cell, comprising administering to said cell the polynucleotide of claim 29, such that said polypeptide mutant is expressed in said cell.

38. A method of diagnosing a condition associated with aberrant eNOS activity, said method comprising:
i) contacting a cell of a patient with said polynucleotide of claim 29; and
ii) detecting a level of eNOS activity indicative of said medical condition.

39. A prophylactic or therapeutic method of treating a condition associated with aberrant eNOS activity, said method comprising administering to a patient in need of treatment an effective amount of said polypeptide mutant of claim 1.

40. A prophylactic or therapeutic method of treating a condition associated with aberrant eNOS activity, said method comprising administering to a patient in need of treatment an effective amount of a polynucleotide encoding said polypeptide mutant of claim 1, such that said polypeptide mutant is expressed in said patient.

41. The method according to claim 39, wherein said method further comprises administering one or more angiogenic factors to said patient before, during, or after said administering of said polypeptide mutant.

42. The method according to claim 40, wherein said method further comprises administering one or more angiogenic factors to said patient, before, during, or after said administering of said polynucleotide.


SEQUENCE LISTING (8 sequences)

SEQ ID NO: 1 - protein, 1203 aa, Homo sapiens
MGNLKSVAQEPGPPCGLGLGLGLGLCGKQGPATPAPEPSRAPASLLPPAPEHSPPSSPLTQPPEGPKFPRVKNWEVGSITYDTLSAQAQQDGPCTPRRCLGSLVFPRKLQGRPSPGPPAPEQLLSQARDFINQYYSSIKRSGSQAHEQRLQEVEAEVAATGTYQLRESELVFGAKQAWRNAPRCVGRIQWGKLQVFDARDCRSAQEMFTYICNHIKYATNRGNLRSAITVFPQRCPGRGDFRIWNSQLVRYAGYRQQDGSVRGDPANVEITELCIQHGWTPGNGRFDVLPLLLQAPDEPPELFLLPPELVLEVPLEHPTLEWFAALGLRWYALPAVSNMLLEIGGLEFPAAPFSGWYMSTEIGTRNLCDPHRYNILEDVAVCMDLDTRTTSSLWKDKAAVEINVAVLHSYQLAKVTIVDHHAATASFMKHLENEQKARGGCPADWAWIVPPISGSLTPVFHQEMVNYFLSPAFRYQPDPWKGSAAKGTGITRKKTFKEVANAVKISASLMGTVMAKRVKATILYGSETGRAQSYAQQLGRLFRKAFDPRVLCMDEYDVVSLEHETLVLVVTSTFGNGDPPENGESFAAALMEMSGPYNSSPRPEQHKSYKIRFNSISCSDPLVSSWRRKRKESSNTDSAGALGTLRFCVFGLGSRAYPHFCAFARAVDTRLEELGGERLLQLGQGDELCGQEEAFRGWAQAAFQAACETFCVGEDAKAAARDIFSPKRSWKRQRYRLSAQAEGLQLLPGLIHVHRRKMFQATIRSVENLQSSKSTRATILVRLDTGGQEGLQYQPGDHIGVCPPNRPGLVEALLSRVEDPPAPTEPVAVEQLEKGSPGGPPPGWVRDPRLPPCTLRQALTFFLDITSPPSPQLLRLLSTLAEEPREQQELEALSQDPRRYEEWKWFRCPTLLEVLEQFPSVALPAPLLLTQLPLLQPRYYSVSSAPSTHPGEIHLTVAVLAYRTQDGLGPLHYGVCSTWLSQLKPGDPVPCFIRGAPSFRLPPDPSLPCILVGPGTGIAPFRGFWQERLHDIESKGLQPTPMTLVFGCRCSQLDHLYRDEVQNAQQRGVFGRVLTAFSREPDNPKTYVQDILRTELAAEVHRVLCLERGHMFVCGDVTMATNVLQTVQRILATEGDMELDEAGDVIGVLRDQQRYHEDIFGLTLRTQEVTSRIRTQSFSLQERQLRGAVPWAFDPPGSDTNSP

SEQ ID NO: 2 - protein, 37 aa, Homo sapiens
AAKGTGITRKKTFKEVANAVKISASLMGTVMAKRVKA

SEQ ID NO: 3 - protein, 37 aa, Bos taurus
ATKGAGITRKKTFKEVANAVKISASLMGTLMAKRVKA

SEQ ID NO: 4 - protein, 38 aa, Homo sapiens
GTNGTPTKRRAIGFKKLAEAVKFSAKLMGQAMAKRVKA

SEQ ID NO: 5 - protein, 38 aa, Rattus rattus; identical to SEQ ID NO: 4

SEQ ID NO: 6 - protein, 37 aa, Rattus rattus
DEKLRPRRREIRFTVLVKAVFFASVLMRKVMASRVRA

SEQ ID NO: 7 - protein, 37 aa, Mus musculus
NEKLRPRRREIRFRVLVKVVFFASMLMRKVMASRVRA

SEQ ID NO: 8 - protein, 37 aa, Homo sapiens
DEKRRPKRREIPLKVLVKAVLFACMLMRKTMASRVRV